(19) United States
(12) Patent Application Publication
(10) Pub. No.: US 2005/0100891 A1
(43) Pub. Date: May 12, 2005

(54) Gene expression profile for KSHV infection and methods for treating same

(76) Inventors: Mattias Luukkonen, San Diego, CA (US); Ashlee V. Moses, Portland, OR (US); Klaus Frueh, Portland, OR (US); Jay A. Nelson, Tualatin, OR (US); Yolanda Corona Bell, Carlsbad, CA (US); Michael Heinrich, Lake Oswego, OR (US); Kenneth Simmen, Tervuren (BE)

Correspondence Address: Phillip A. Singer;WOODCOCK WASHBURN LLP, 46th Floor, One Liberty Place, Philadelphia, PA, 19103, US

(21) Appl. No.: 11/019,987

(22) Filed: Dec. 23, 2004

Related U.S. Application Data
(62) Division of application No. 09/921,512, filed on Aug. 1, 2001.
(60) Provisional application No. 60/222,162, filed on Aug. 1, 2000.

(51) Int. Cl. C12Q 1/70; C12Q 1/68; C12M 1/34; A61K 38/16

(52) U.S. Cl. 435/5; 435/6; 435/287.2; 514/2

(57) ABSTRACT

The present invention utilizes nucleic acid microarray technology to identify changes in the host endothelial cell transcription pattern that occurs during the latent and lytic phase of the KSHV life cycle. The production or activity of some genes up regulated during the lytic cycle were subsequently inhibited, and two such targets were shown to have a role in expression of late viral genes. Using this combined approach we have identified cellular pathways previously unknown to be important for KSHV infection, and present evidence for the efficiency of the novel antiviral approaches thus discovered. In addition, the present invention identifies a wide variety of endothelial cells genes and pathways that are involved in a variety of endothelial cell-mediated activity, including angiogenesis and transformation.

### CROSS-REFERENCE TO RELATED APPLICATIONS

This application is a divisional of U.S. patent application Ser. No. 09/921,512, filed Aug. 1, 2001, now pending, which application claims the benefit of U.S. Provisional Application Ser. No. 60/222,162, filed 2 Aug. 2000, which applications are incorporated herewith in their entireties.

### BACKGROUND OF THE INVENTION

Kaposi's Sarcoma (KS) is a vascular neoplasm characterized by angioproliferative lesions on the skin and visceral organs. The lesions are distinguished by the presence of proliferating spindle-shaped tumor cells of endothelial origin and exhibit abnormal vascularization with extensive extravasation of inflammatory cells and erythrocytes. While KS is a rare condition in immunocompetent individuals, it is the most common malignancy associated with acquired immunodeficiency syndrome (AIDS). Human herpesvirus 8 (HHV8) or Kaposi's sarcoma-associated herpesvirus (KSHV) is the infectious agent associated with KS development⁽¹⁴⁾. The virus infects the majority of spindle cells in the lesion, as well as lesional endothelial cells and infiltrating leukocytes^{(78, 79, 80, 81)}. The majority of spindle cells harbor the KSHV genome in latent form with a small percentage of cells entering lytic cycle and producing infections virus^{(72, 82, 80, 81)}. The exact role of latent and lytic virus gene expression in the complex biology of KSHV has yet to be fully elucidated. In addition, little is known about the influence of virus gene expression on cellular gene profiles or how these virus-cell interactions contribute to tumorigenesis. The establishment of valid in vitro culture systems should allow resolution of many questions associated with KSHV and KS. We recently developed such a culture system using immortalized dermal microvascular endothelial cells that are extremely permissive for KSHV infection and maintain the genome indefinitely in a primarily latent state. Infection of DMVEC with KSHV leads to phenotypic changes including spindle cell formation, loss of contact inhibition and growth in soft agar, suggesting virus-cell interactions that mimic those driving KS lesion formation in vivo.

The changes induced by KSHV infection are reflected in an altered transcriptional program of the host cell. DNA microarray analysis enables the contemporaneous monitoring of transcriptional changes for several thousands of host genes⁽⁶⁰⁾. Automated quantitation of RNA samples hybridized to glass slides imprinted with known DNA fragments allows for comparison of transcriptional differences under various experimental conditions.

### SUMMARY OF THE INVENTION

Kaposi's Sarcoma-associated herpesvirus infection of human endothelial cells leads to spindle cell formation and malignant transformation. DNA microarray analysis demonstrated that virus infection caused profound changes of multiple cellular pathways including those governing angiogenesis, inflammation and differentiation. Upon induction of the lytic cycle, KSHV further modulated the host cell response. To evaluate whether genes overexpressed during the lytic cycle constitute potential antiviral targets, we systematically inhibited their induction or function. Haloperidol, an antagonist of the type I Sigma receptor, and Ht-31, a peptide inhibitor of the interaction between kinase anchoring proteins (AKAP) and protein kinase A (PKA), inhibited expression of lytic, but not latent viral gene expression. One of the genes significantly up regulated following KSHV infection was the gene for the c-Kit (CD117) protein, a receptor tyrosine kinase that functions as the receptor for stem cell factor (steel factor, SCF)^{(83, 84)}. KSHV-infected DMVEC exhibit an enhanced proliferative responsive to exogenous SCF and inhibition of c-Kit signalling leads to a reduction in ligand-dependent proliferation and reverses the KSHV-induced transformed phenotype. Furthermore, inhibition of c-kit signal transduction prevents KSHV-induced spindle cell formation whereas adenovirus vector-mediated expression of c-kit induces spindle cell formation in uninfected endothelial cells. Thus, KSHV contributes to KS development through modulation of c-Kit expression and function. The present invention provides methods and compositions for treating and diagnosing KSHV infection, as well as Kaposi's Sarcoma. The present invention also provides the gene expression profile for KSHV infected cells at various stages of viral replication. In addition, the present invention provides methods and compositions for the treatment and diagnosis of endothelial cell transformation. The present invention also provides methods and compositions for modulating angiogenesis, including to stimulate or inhibit neo-vascularization, as well as modulating the extracellular matrix surrounding endothelial cells. The present invention provides methods and compositions for the modulation of interferon mediated gene expression. Additionally, the present invention provides methods and compositions for the modulation of endothelial cell inflammation. Also included in the present invention are methods and compositions for the dedifferentiation of endothelial cells. The present invention is also directed to methods of using said gene expression profiles and microarrays, and business methods directed to said use of gene expression profiles and microarrays.

### BRIEF DESCRIPTION OF THE DRAWING

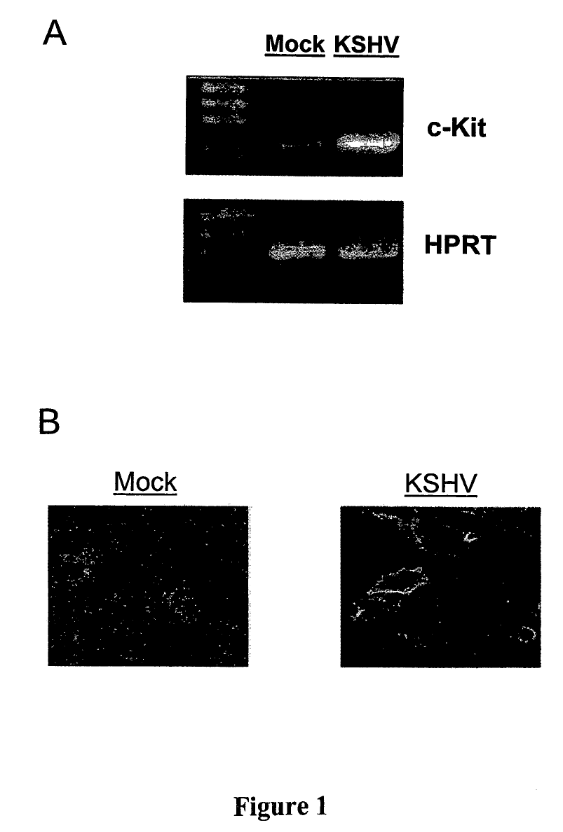

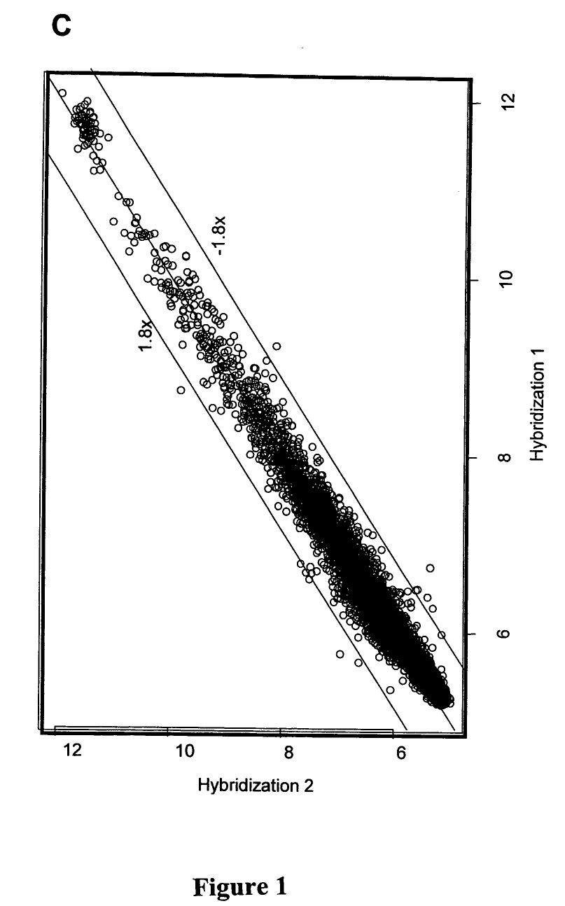

FIG. 1, Panels A, B and C: Induction of c-Kit mRNA and protein expression on DMVEC following KSHV infection.
A) RT-PCR for c-Kit mRNA using reverse transcribed total RNA prepared from mock- (Mock) and KSHV-infected (KSHV) DMVEC. RT-PCR reactions were performed using primers designed to amplify a 242 bp fragment of c-Kit. RT-PCR for HPRT was performed as a control for each sample. Amplification products were visualized by ethidium bromide staining of agarose gels,
B) Immunofluorescence microscopy showing up-regulation of c-Kit protein on the surface of KSHV-infected (KSHV) DMVEC. Low levels of constitutive c-Kit expression were detected on the surface of mock-infected cells (Mock),
C) reproducibility of microarray analysis of human gene expression. Scatter plot analysis showing correlation between 2 independent microarray hybridizations of RNA isolated from uninfected cells. The fluorescence intensities are shown for each experiment.

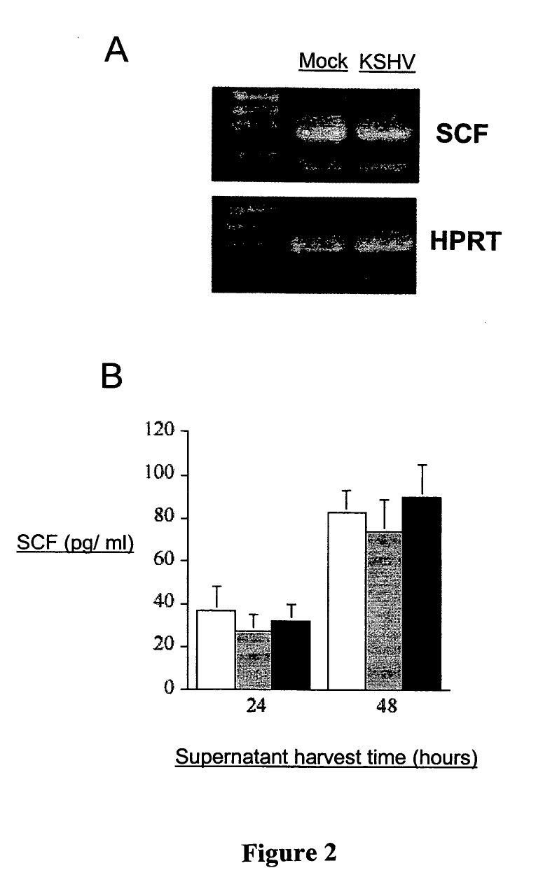

FIG. 2, Panels A and B: DMVEC production of SCF is not affected by KSHV infection.
A) RT-PCR for SCF mRNA using reverse transcribed total RNA prepared from mock- (Mock) and KSHV-infected (KSHV) DMVEC. RT-PCR reactions were performed using primers designed to amplify a 274-bp fragment of SCF. RT-PCR for HPRT was performed as a control for each sample. Amplification products were visualized by ethidium bromide staining of agarose gels.
B) Soluble SCF (pg/ml) in supernatant conditioned by mock-infected DMVEC (open bars) and two different lines of KSHV-infected DMVEC (hatched and solid bars) for 24 and 48 hours. SCF levels were quantitated by ELISA. Results from triplicate samples (±SD) are shown.

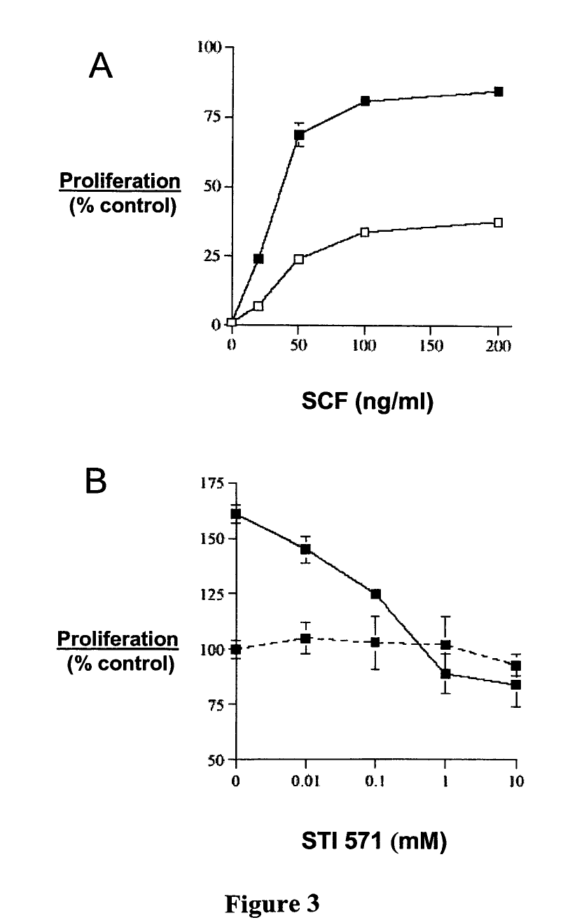

FIG. 3, Panels A and B: SCF-dependent proliferation of DMVEC is enhanced by KSHV infection.
A) Proliferative response of mock- (open squares) and KSHV-infected (filled squares) DMVEC to exogenous SCF. Proliferation was measured using an XTT-based assay (see Methods). Results from triplicate wells (±SD) are expressed as the percentage increase over basal proliferation measured in the absence of exogenous SCF. Representative results from 1 of 3 independent experiments are shown.
B) An inhibitor of c-Kit tyrosine kinase activity (STI 571) abrogates SCF-dependent DMVEC proliferation. KSHV-infected DMVEC were cultured in the presence of increasing doses of STI 571 and in the presence (filled circles) or absence (open circles) of exogenous SCF (50 ng/ml). Proliferation of KSHV-infected DMVEC was measured with an XTT-based assay as described above but were added at the same time as SCF (50 ng/ml). Results for triplicate wells (±SD) are expressed as a percentage of basal proliferation measured in the absence of SCF and STI 571 (expressed as 100%). Representative results from 1 of 3 independent experiments are shown.

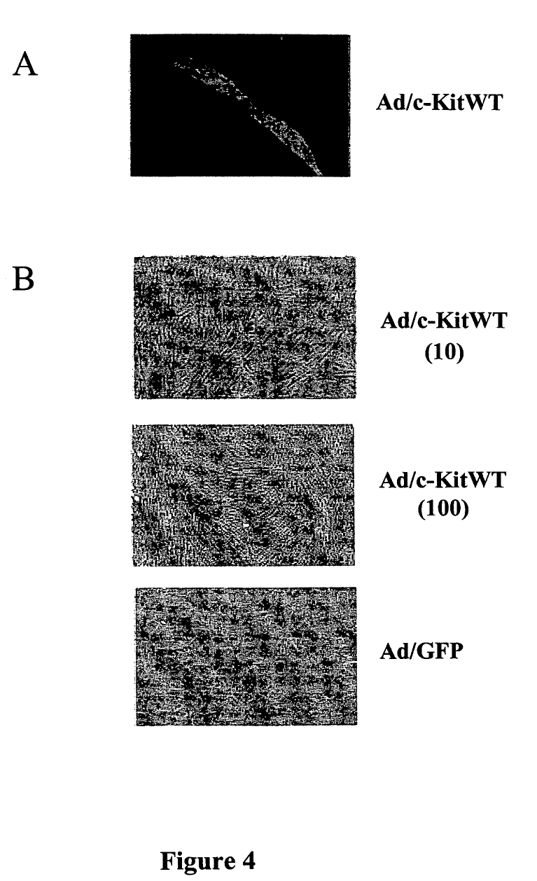

FIG. 4, Panels A and B: Ectopic expression of c-Kit in normal DMVEC induces morphological changes.
A) Light microscopy illustrating the dose-dependent development of spindle morphology and disorganized growth in normal DMVEC following infection with an adenovirus vector expressing c-Kit (Ad/c-KitWT) at an MOI of 10 or 100. DMVEC infected with Ad/GFP at an MOI of 100 (Ad/GFP) maintained a normal cobblestone morphology.
B) Immunofluorescence microscopy showing strong expression of c-Kit protein specifically on DMVEC that developed spindle morphology following infection with Ad/c-KitWT.

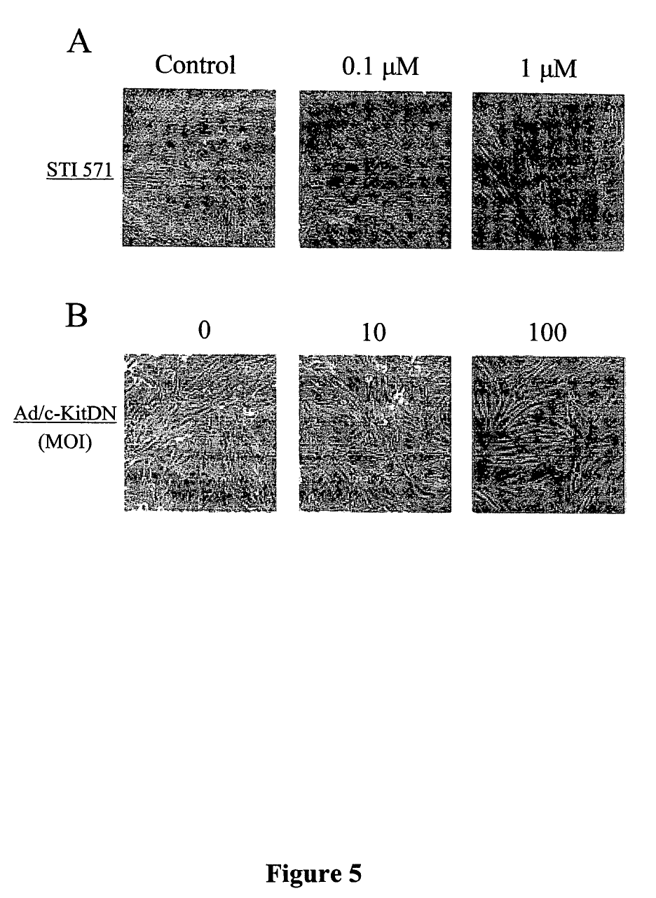

FIG. 5, Panels A and B: Inhibition of c-Kit tyrosine kinase activity reverses the transformed phenotype of KSHV-infected DMVEC.
A) KSHV-infected DMVEC exhibiting disorganized growth and focus formation were left untreated (Control) or treated with STI 571 (0.1 and 1 μM) for 5 days. The STI 571-induced foci loss and monolayer re-organization was observed and recorded with a Nikon light microscope.
B) KSHV-infected DMVEC exhibiting disorganized growth and focus formation were left untreated (0), or infected with an adenovirus vector expressing a dominant negative c-Kit protein (Ad/c-KitDN) at an MOI of 10 or 100 for 5 days and the abrogation of focus formation recorded as described above. For both the pharmacological and dominant negative protein inhibition protocols, results from one of three independent experiments are shown. Fields photographed were representative of the entire monolayer of treated cells.

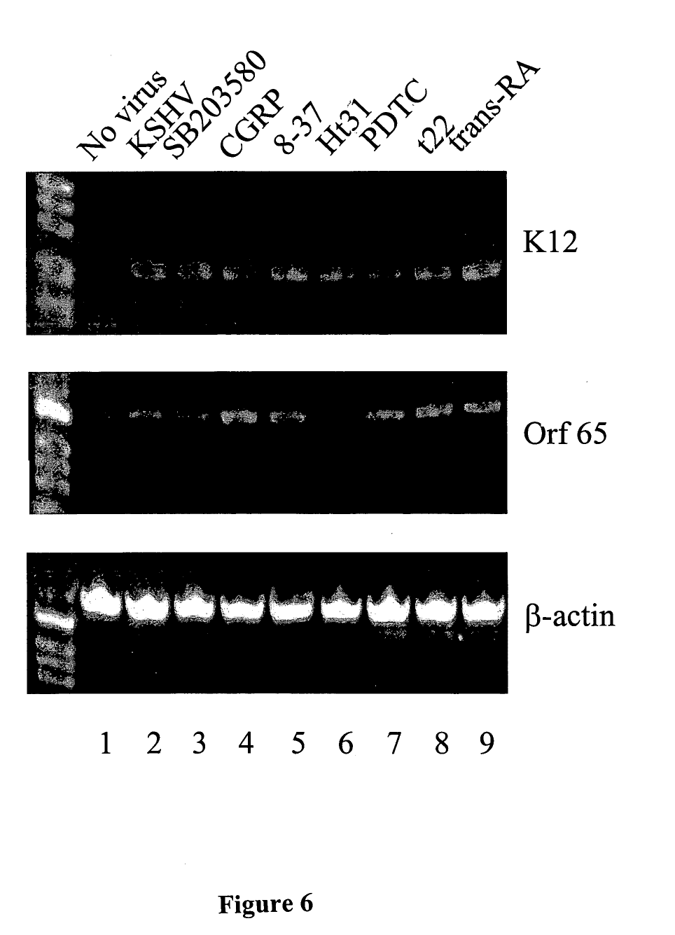

FIG. 6: Inhibition of KSHV late gene expression.
KSHV-infected DMVEC were induced with phorbol esters, and treated with the indicated compounds for 48 hours prior to RNA isolation and RT-PCR. Concentration for compounds were 5 μM. Primers specific for K12 (latent gene) or ORF 65 (lytic gene), the cellular gene HPRT of beta-actin were used for RT PCR.

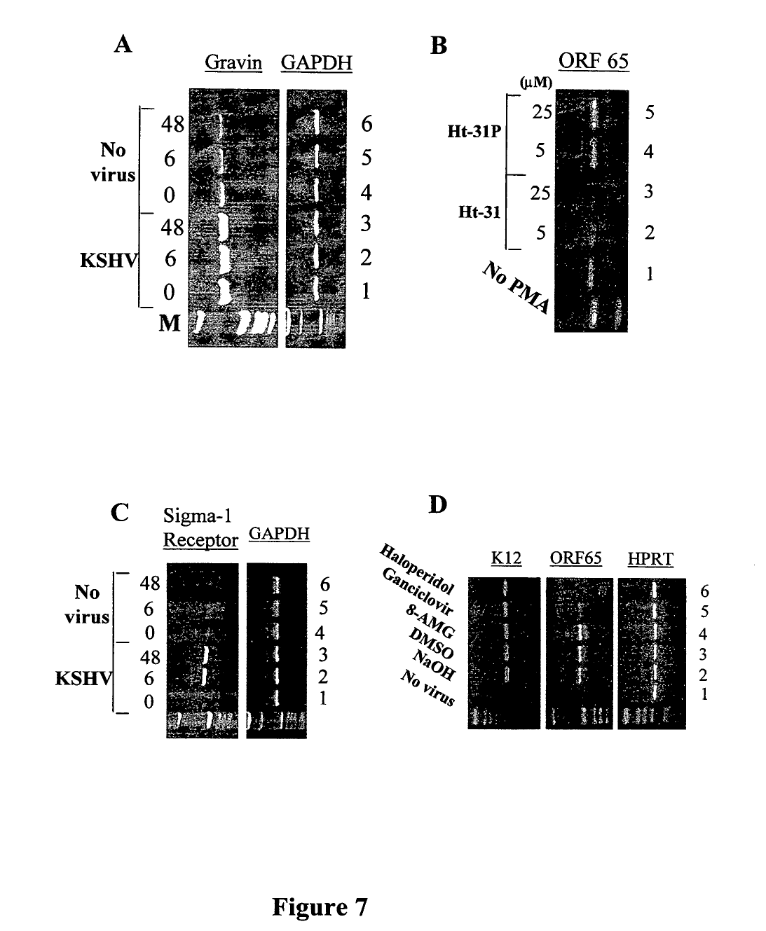

FIG. 7, Panels A, B, C and D: The AKAP/PKA interaction, and the type I Sigma receptor are required for late gene expression.
A) RT-PCR analysis of Gravin expression in uninfected and KSHV infected endothelial cells. Cells were treated with PMA for the times indicated;
B) RT-PCR analysis of ORF65 gene expression after treatment with the AKAP/PKA inhibitory peptide Ht-31 or control peptide Ht-31P at indicated concentrations;
C) RT-PCR analysis of the type I Sigma receptor expression in uninfected cells and cells infected with KSHV. Cells were treated with PMA for the times indicated. The cellular GAPDH gene was amplified as control for the cDNA synthesis;
D) RT-PCR analysis of latent (K12) or late (ORF 65) gene expression after treatment with various inhibitory compounds. Final drug concentrations were as follows: 5 uM Haloperidol, 10 μM 8-aminoguanosine and 25 uM Ganciclovir. The cellular HPRT gene was amplified as control for the cDNA synthesis.

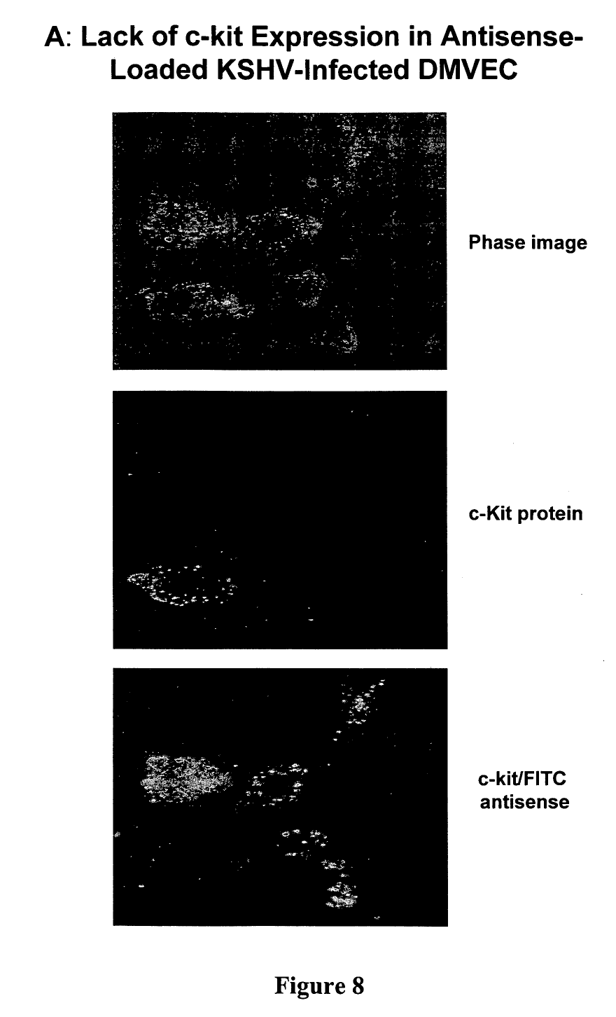

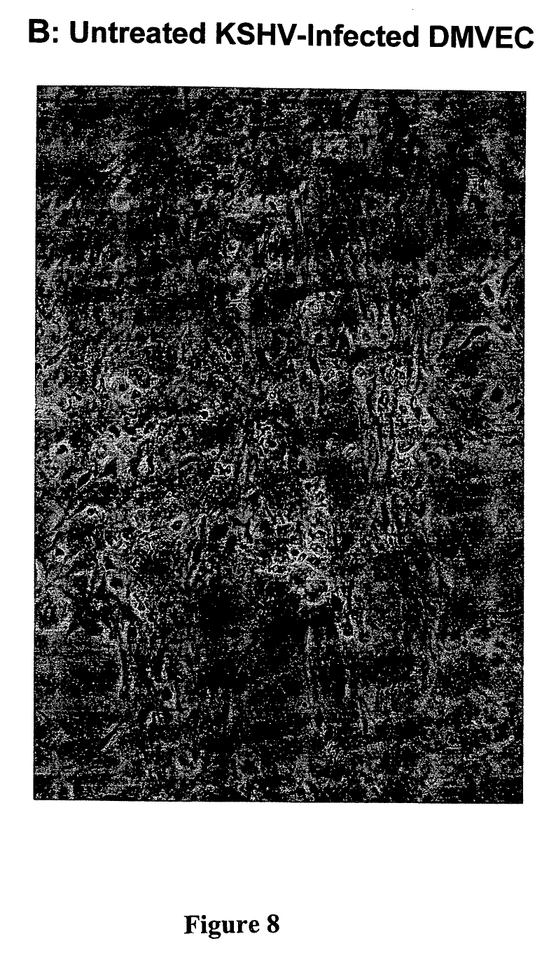

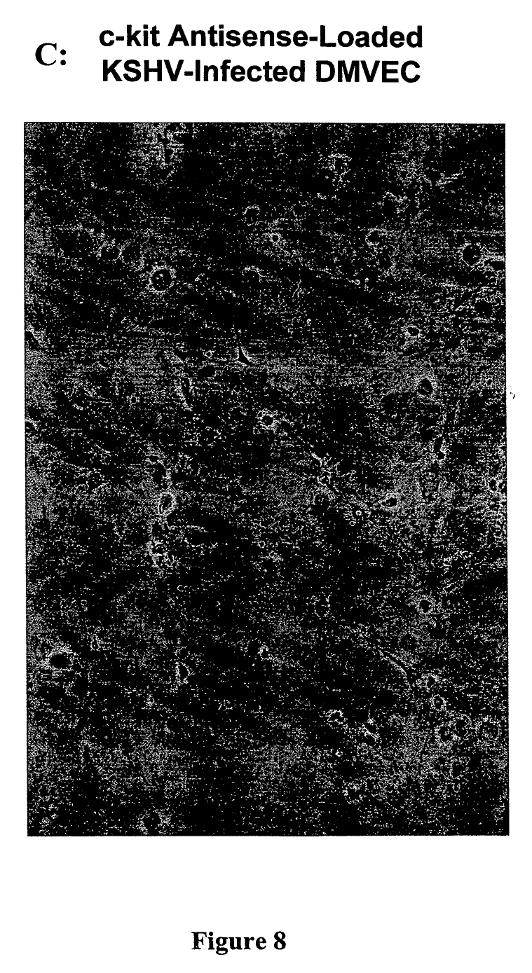

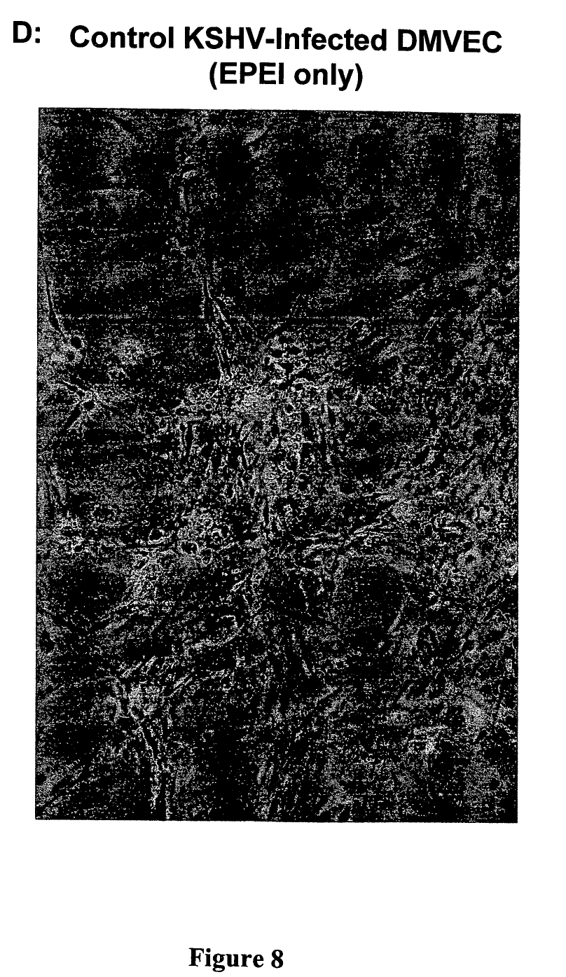

FIG. 8, Panels A, B, C and D: A c-kit antisense oligomer inhibits c-kit protein expression and prevents development of the transformed phenotype in KSHV-infected DMVEC.
A) Immunofluorescence microscopy performed on a KSHV-infected DMVEC monolayer demonstrating inhibition of c-kit expression in cells that have taken up a FITC-tagged c-kit antisense oligomer (green), relative to the strong c-Kit expression, visualized by binding of a goat anti-mouse Alexa 594 secondary antibody (red), of antisense-negative (FITC negative) cells;
B) Focus formation in KSHV-infected DMVEC cultured post confluency;
C) Inhibition of focus formation in KSHV-infected DMVEC treated with a c-kit-antisense oligomer at confluence;
D) Focus formation in control KSHV-infected DMVEC exposed to the EPEI delivery reagent at confluence.

### DETAILED DESCRIPTION

The analysis of host cell-virus interactions by DNA microarrays allows for detection of previously unknown relationships between cellular pathways and viral infection. By employing this technology to endothelial cells harboring KSHV, we identified cellular genes whose expression is regulated by KSHV. One of the cellular transcripts that was consistently upregulated in all three experiments was c-kit. C-kit is a receptor tyrosine kinase activated by stem cell factor (SCF). c-Kit over expression following KSHV infection affects both paracrine and autocrine c-Kit/SCF interactions. The enhanced expression with functional consequences raises the possibility that c-Kit expression may play a role in the development of KS. KSHV-infection of DMVEC enhances expression of the SCF receptor, c-Kit, and increased expression promotes enhanced proliferation in response to exogenous SCF. Further, DMVEC co-express the c-Kit ligand SCF, and that inhibition of autocrine c-Kit tyrosine kinase activity inhibits the transformed phenotype induced in DMVEC by KSHV infection. While c-Kit expression and c-Kit/SCF interactions are crucial for the normal development of hematopoietic cells and a restricted number of non-hematopoietic cells, expression of c-Kit has also been associated with various malignancies. In c-Kit-associated cancers, different roles for c-Kit have been identified. For example, in GISTs and germ cell neoplasms, gain-of function mutations in juxtamembrane and tyrosine kinase domains have been described, permitting, respectively, ligand-independent dimerization or constitutive activation without dimerization^{(85, 86, 87, 88)}. In small cell lung carcinoma (SCLC) and breast cancer, co-expression of c-Kit and SCF occurs and is thought to generate an autocrine growth loop^{(89, 90, 91, 92, 93)}. Since endothelial cells are the precursors of KS spindle cells, our finding that KSHV infection of dermal endothelial cells enhances c-Kit expression with functional consequences raises the possibility that c-Kit expression may play a role in the development of KS. A recent report documented for the first time expression of c-Kit in KS tissue⁽⁹⁴⁾ and, in one case examined in more detail, no activating mutations in juxtamembrane or tyrosine kinase domains were observed. This in vivo finding supports that c-Kit plays a role in the pathogenesis of KS.

During KS disease, the presence of KSHV in atypical endothelial cells in KS lesions suggests a role for KSHV in spindle cell development and tumor progression. In our study, the observation that KSHV infection of DMVEC up-regulates functional c-Kit receptors, combined with the demonstration that c-Kit activity is required for cellular transformation, strongly supports a role for c-Kit/SCF in the development of KS.

Identification of a causative role for c-Kit in KSHV-associated cellular transformation suggests a novel therapeutic target for KS. STI 571, the c-Kit tyrosine kinase inhibitor described in this report, has shown considerable promise in phase I/II clinical trials for the treatment of chronic myelogenous leukemia⁽⁹⁵⁾, a disease associated with Bcr-Abl kinase activity. Recently, STI 571 was also shown to inhibit the growth of SCLC cell lines in vitro via inhibition of c-Kit activity^{(93, 96)}. Over 70% of SCLC co-express c-Kit and SCF, suggesting that inhibitors of c-Kit activity may have clinical application for treatment of this and other cancers in which autocrine c-Kit signaling plays a role. In conclusion, c-Kit should be considered a primary target in KS tumorigenesis. Consequently, STI 571, or other pharmacological inhibitors of c-Kit signaling, should be evaluated as potential therapeutic agents for the treatment of KS.

STI 571 (GLEEVEC®), the pharmacological inhibitor of c-Kit tyrosine kinase activity described in this report, is a 2-phenylaminopyrimidine derivative that was originally selected for specificity against the Abl tyrosine kinase⁽⁹⁷⁾. This inhibitor was subsequently shown to display an extended but narrow range of specificity, being active against the Bcr-Abl fusion protein, platelet-derived growth factor receptor and c-kit receptor tyrosine kinases^{(97, 98, 99)}. The Bcr-Abl fusion gene is found in 95% of patients with chronic myelogenous leukemia (CML) and the enhanced tyrosine kinase activity of Bcr-Abl is thought to be a major factor in the disease. STI 571 inhibits the proliferation and tumor formation of Bcr-Abl-expressing cells⁽⁹⁸⁾ and been approved for the treatment of CML⁽⁹⁵⁾. Our results show that STI 571, as well as other inhibitors of c-Kit signalling have therapeutic potential for the treatment of KS and in inhibiting the replication of KSHV.

We also identified cellular genes required for viral gene expression. By subtracting all genes expressed in non-induced cells as well as those induced in non-infected cells, we identified a small group of genes specifically and significantly induced by KSHV 6 hours or 48 hours after PMA stimulation. A large fraction of this group contained genes induced by pro-inflammatory stimuli and thus most likely regulated by NFkB. Induction of pro-inflammatory genes during lytic replication could represent an innate immune response of the host, or could be caused by viral activation of NF-kB. Consistently, inhibition of NFkB activation induced KSHV-mediated apoptosis in B cells. However, this could not be confirmed with the small molecule NFkB inhibitor used in this study. The function or induction of 23 genes were directly targeted by inhibitors. Of these, two targets were found to be required for late gene expression. Thus, a significant fraction of the up-regulated genes are essential for viral gene expression, and a less stringent search would reveal additional genes required for the viral life cycle. For instance, expanding the pool of putative target genes to those induced during latency may reveal cellular gene products essential for tumor cell proliferation or reactivation of the latent provirus. Therefore, the DNA chip represents a powerful tool to rapidly identify potential cellular drug targets.

We have identified the AKAP/PKA interaction, and the type I Sigma receptor as essential cellular components for expression of the ORF 65 gene. PKA is a ubiquitous signaling component activated by intracellular cAMP. The association with various AKAPs mediates intracellular localization of PKA, and ensures timely activation in response to extracellular stimuli^{(21, 26)}. PKA has been found associated with cell surface receptors like the NMDA and Glutamate receptors, and with intracellular structures such as mitochondria^{(21, 26)}. The AKAP Yotiao tethers PKA to protein phosphatase 1 and the NMDA receptor, and PKA activation can thereby modulate the activity of ion channels⁽⁷⁰⁾. In addition, PKA controls transcription by phosphorylation of nuclear factors like the cAMP-response element binding protein⁽⁶³⁾.

The type I Sigma receptor was characterized as the binding site for anti-psychotic drugs such as Haloperidol⁽³³⁾. The receptor is localized to the ER, and may translocate to the cell surface upon activation⁽⁴²⁾. Like PKA, the type I Sigma receptor has been implicated in control of ion channel activity. For instance, type I Sigma agonists affect NMDA-induced calcium signaling in neurons^{(28, 32)}. It has been demonstrated that the type I Sigma receptor affected both IP3 mediated calcium release from the ER, and extracellular calcium influx after membrane depolarization⁽²⁹⁾. Interestingly, PKA phosphorylation of the IP3 receptor increases its sensitivity⁽⁷⁾, suggesting a common site of action for the drug targets identified. Little is known about the importance of calcium signaling during KSHV infection, however it has been reported that ionomycin strongly augmented the reactivation process induced by phorbol esters⁽¹³⁾. Taken together, this suggests calcium signaling may be required for efficient reactivation or replication of KSHV. Interestingly, Haloperidol has been shown to inhibit replication of another gamma-herpesvirus, the Epstein-Barr Virus (EBV), in tissue culture⁽⁴⁹⁾. Experiments to further investigate the requirements for calcium signaling in gamma-herpesvirus reactivation and replication are currently underway.

Our approach represents a novel strategy for anti-viral research. Different from traditional, hypothesis-driven approaches, we used a data-driven approach to identify potential targets for intervention with the lytic cycle of KSHV. From the large body of data obtained by global transcriptional analysis of virus-infected host cells we selected genes that met certain predetermined criteria. By inhibiting either upstream regulators or the function of most of the proteins encoded by these genes, host cell pathways important for viral replication can be identified. These pathways offer novel routes of intervention, or even novel applications for known drugs. Thus, DNA-microarrays can be applied to the development of novel treatments for viral diseases.

The present invention relates to nucleic acid molecules and the polypeptides encoded thereby, that are identified in Table 2, which represent the endothelial cell genes whose expression is modulated by KSHV infection. The complete amino acid sequence of the KSHV modulated endothelial cell genes may or may not be known, and the complete nucleotide sequence encoding the full length genomic DNA or the amino acid coding region may or may not be known. It is predicted that a wide variety of mammalian cells and cell types of endothelial cell lineage that are readily available from depositions such as The American Type Culture Collection, will contain the described genes. Endothelial cells of human origin capable of producing one or more of the KSHV modulated endothelial cell genes include, but are not limited to HUV-EC-C (CRL-1730), HAAE-1 (CRL-2472), HAAE-2 (CRL-2473), HFAE-2 (CRL-2474), HIAE-78 (CRL-2475), HIAE-101 (CRL-2478), HVVE-12 (CRL-2480) and DMVEC.

Other cells and cell lines may also be suitable for use to isolate KSHV modulated endothelial cell genes cDNA. Selection of suitable cells may be done by screening for KSHV modulated endothelial cell gene expression activity in cell extracts or in whole cell assays. Cells that possess KSHV modulated endothelial cell gene activity in any one of these assays may be suitable for the isolation of KSHV modulated endothelial cell gene DNA or mRNA.

Any of a variety of procedures known in the art may be used to molecularly clone KSHV modulated endothelial cell gene DNA. These methods include, but are not limited to, direct functional expression of the KSHV modulated endothelial cell genes following the construction of a cDNA library from appropriate cells in an appropriate expression vector system. Another method is to screen a cDNA library constructed in a bacteriophage or plasmid shuttle vector with a labelled oligonucleotide probe designed from the amino acid sequence encoded by one or more of the KSHV modulated endothelial cell genes. An additional method consists of screening a cDNA library constructed in a bacteriophage or plasmid shuttle vector with a partial cDNA encoding the protein encoded by a KSHV modulated endothelial cell gene. This partial cDNA is obtained by the specific PCR amplification of DNA fragments through the design of degenerate oligonucleotide primers from the amino acid sequence of the purified protein encoded by one or more KSHV modulated endothelial cell genes.

Another method is to isolate RNA from cells and translate the RNA into protein via an in vitro or an in vivo translation system. The translation of the RNA into a peptide a protein will result in the production of at least a portion of the protein of interest which can be identified by, for example, immunological reactivity with an anti-protein antibody or by biological activity of the protein. In this method, pools of RNA isolated from appropriate cells can be analyzed for the presence of an RNA that encodes at least a portion of the desired protein. Further fractionation of the RNA pool can be done to purify the appropriate RNA from unwanted RNA. The peptide or protein produced by this method may be analyzed to provide amino acid sequences which in turn are used to provide primers for production of KSHV modulated endothelial cell gene cDNA, or the RNA used for translation can be analyzed to provide nucleotide sequences encoding KSHV modulated endothelial cell gene products and produce probes for this production of cDNA. This method is known in the art and can be found in, for example, Maniatis, T., Fritsch, E. F., Sambrook, J. in Molecular Cloning: A Laboratory Manual, Second Edition, Cold Spring Harbor Laboratory Press, Cold Spring Harbor, N.Y. 1989.

It is readily apparent to those skilled in the art that other types of libraries, as well as libraries constructed from other cells or cell types, may be useful for isolating KSHV modulated endothelial cell genes-encoding DNA. Other types of libraries include, but are not limited to, cDNA libraries derived from other cells, and genomic DNA libraries that include YAC (yeast artificial chromosome) and cosmid libraries.

It is readily apparent to those skilled in the art that suitable cDNA libraries may be prepared from cells or cell lines which have KSHV modulated endothelial cell gene activity. The selection of cells or cell lines for use in preparing a cDNA library to isolate KSHV modulated endothelial cell gene cDNA may be done by first measuring cell associated biological activity using the measurement of the appropriate biological activity or a ligand binding assay.

Preparation of cDNA libraries can be performed by standard techniques well known in the art. Well known cDNA library construction techniques can be found for example, in Maniatis, T., Fritsch, E. F., Sambrook, J., Molecular Cloning: A Laboratory Manual, Second Edition (Cold Spring Harbor Laboratory, Cold Spring Harbor, N.Y., 1989).

It is also readily apparent to those skilled in the art that DNA encoding KSHV modulated endothelial cell genes may also be isolated from a suitable genomic DNA library. Construction of genomic DNA libraries can be performed by standard techniques well known in the art. Well known genomic DNA library construction techniques can be found in Maniatis, T., Fritsch, E. F., Sambrook, J. in Molecular Cloning: A Laboratory Manual, Second Edition (Cold Spring Harbor Laboratory, Cold Spring Harbor, N.Y., 1989).

In order to clone the KSHV modulated endothelial cell genes by the above methods, the amino acid sequence of the gene product may be necessary. To accomplish this, protein may be purified and partial amino acid sequence determined by automated sequenators. It is not necessary to determine the entire amino acid sequence, but the linear sequence of two regions of 6 to 8 amino acids from the protein is determined for the production of primers for PCR amplification of a partial KSHV modulated endothelial cell gene DNA fragment.

Once suitable amino acid sequences have been identified, the DNA sequences capable of encoding them are synthesized. Because the genetic code is degenerate, more than one codon may be used to encode a particular amino acid, and therefore, the amino acid sequence can be encoded by any of a set of similar DNA oligonucleotides. Only one member of the set will be identical to the KSHV modulated endothelial cell gene sequence but will be capable of hybridizing to DNA even in the presence of DNA oligonucleotides with mismatches. The mismatched DNA oligonucleotides may still sufficiently hybridize to the KSHV modulated endothelial cell gene DNA to permit identification and isolation of KSHV modulated endothelial cell gene encoding DNA. DNA isolated by these methods can be used to screen DNA libraries from a variety of cell types, from invertebrate and vertebrate sources, and to isolate homologous genes.

Because the genetic code is degenerate, more than one codon may be used to encode a particular amino acid, and therefore, the amino acid sequence can be encoded by any of a set of similar DNA oligonucleotides. Only one member of the set will be identical to the KSHV modulated endothelial cell gene sequence but will be capable of hybridizing to KSHV modulated endothelial cell gene nucleic acids even in the presence of DNA oligonucleotides with mismatches under appropriate conditions. Under alternate conditions, the mismatched DNA oligonucleotides may still hybridize to the KSHV modulated endothelial cell gene DNA or RNA to permit identification and/or isolation of KSHV modulated endothelial cell gene encoding DNA.

Recombinant Expression of KSHV Modulated Endothelial Cell Genes

The cloned KSHV modulated endothelial cell gene DNA obtained through the methods described herein may be recombinantly expressed by molecular cloning into an expression vector containing a suitable promoter and other appropriate transcription regulatory elements, and transferred into prokaryotic or eukaryotic host cells to produce recombinant KSHV modulated endothelial cell gene protein. Techniques for such manipulations are fully described in Maniatis, T, et al., supra, and are well known in the art.

Expression vectors are defined herein as DNA sequences that are required for the transcription of cloned copies of genes and the translation of their mRNAs in an appropriate host. Such vectors can be used to express eukaryotic genes in a variety of hosts such as bacteria including E. coli, blue-green algae, plant cells, insect cells, fungal cells including yeast cells, and animal cells.

Specifically designed vectors allow the shuttling of DNA between hosts such as bacteria-yeast or bacteria-animal cells or bacteria-fungal cells or bacteria-invertebrate cells. An appropriately constructed expression vector should contain: an origin of replication for autonomous replication in host cells, selectable markers, a limited number of useful restriction enzyme sites, a potential for high copy number, and active promoters. A promoter is defined as a DNA sequence that directs RNA polymerase to bind to DNA and initiate RNA synthesis. A strong promoter is one that causes mRNAs to be initiated at high frequency. Expression vectors may include, but are not limited to, cloning vectors, modified cloning vectors, specifically designed plasmids or viruses.

A variety of mammalian expression vectors may be used to express recombinant KSHV modulated endothelial cell genes in mammalian cells. Commercially available mammalian expression vectors which may be suitable for recombinant KSHV modulated endothelial cell genes expression, include but are not limited to, pMAMneo (Clontech), pcDNA3 (Invitrogen), pMC1neo (Stratagene), pXT1 (Stratagene), pSG5 (Stratagene), EBO-pSV2-neo (ATCC 37593) pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565).

A variety of bacterial expression vectors may be used to express recombinant KSHV modulated endothelial cell genes in bacterial cells. Commercially available bacterial expression vectors which may be suitable for recombinant KSHV modulated endothelial cell gene expression include, but are not limited to pET vectors (Novagen) and pQE vectors (Qiagen).

A variety of fungal cell expression vectors may be used to express recombinant proteins in fungal cells such as yeast. Commercially available fungal cell expression vectors which may be suitable for recombinant KSHV modulated endothelial cell gene expression include but are not limited to pYES2 (Invitrogen) and Pichia expression vector (Invitrogen).

A variety of insect cell expression vectors may be used to express recombinant KSHV modulated endothelial cell genes in insect cells. Commercially available insect cell expression vectors which may be suitable for recombinant expression of KSHV modulated endothelial cell genes include but are not limited to pBlueBacII (Invitrogen).

DNA encoding KSHV modulated endothelial cell genes may be cloned into an expression vector for expression in a recombinant host cell. Recombinant host cells may be prokaryotic or eukaryotic, including but not limited to bacteria such as E. coli, fungal cells such as yeast, mammalian cells including but not limited to cell lines of human, bovine, porcine, monkey and rodent origin, and insect cells including but not limited to drosophila and silkworm derived cell lines. Cell lines derived from mammalian species which may be suitable and which are commercially available, include but are not limited to, CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26), MRC-5 (ATCC CCL 171), L-cells, and HEK-293 (ATCC CRL1573).

The expression vector may be introduced into host cells via any one of a number of techniques including but not limited to transformation, transfection, protoplast fusion, lipofection, and electroporation. The expression vector-containing cells are clonally propagated and individually analyzed to determine whether they produce protein encoded by the KSHV modulated endothelial cell genes. Identification of KSHV modulated endothelial cell gene expressing host cell clones may be done by several means, including but not limited to immunological reactivity with antibodies, and the presence of host cell-associated protein biological activity.

Expression of KSHV modulated endothelial cell gene DNA may also be performed using in vitro produced synthetic mRNA. Synthetic mRNA or mRNA isolated from the appropriate cells can be efficiently translated in various cell-free systems, including but not limited to wheat germ extracts and reticulocyte extracts, as well as efficiently translated in cell based systems, including but not limited to microinjection into frog oocytes, with microinjection into frog oocytes being generally preferred.

Assay Methods for KSHV Modulated Endothelial Cell Genes

Host cell transfectants and microinjected oocytes may be used to assay both the levels of functional KSHV modulated endothelial cell gene activity and levels of total protein encoded by KSHV modulated endothelial cell genes by the following methods. In the case of recombinant host cells, this involves the co-transfection of one or possibly two or more plasmids, containing the DNA encoding one or more fragments or subunits of the proteins encoded by KSHV modulated endothelial cell genes. In the case of oocytes, this involves the co-injection of synthetic RNAs for protein encoded by the KSHV modulated endothelial cell genes. Following an appropriate period of time to allow for expression, cellular protein is metabolically labelled with, for example ³⁵S-methionine for 24 hours, after which cell lysates and cell culture supernatants are harvested and subjected to immunoprecipitation with polyclonal antibodies directed against the protein.

Levels of the appropriate protein in host cells are quantitated by immunoaffinity and/or ligand affinity techniques. Cells expressing KSHV modulated endothelial cell genes can be assayed for the number of protein molecules expressed by measuring the amount of radioactive [ligand] binding to cell membranes. Protein-specific affinity beads or protein-specific antibodies are used to isolate for example ³⁵S-methionine labelled or unlabelled protein. Labelled protein is analyzed by SDS-PAGE. Unlabelled protein is detected by Western blotting, ELISA or RIA assays employing protein-specific antibodies.

Other methods for detecting KSHV modulated endothelial cell gene activity involve the direct measurement of KSHV modulated endothelial cell gene encoded protein activity in whole cells transfected with cDNA or oocytes injected with mRNA. The desired protein activity is measured by specific ligand binding or biological characteristics of the host cells expressing KSHV modulated endothelial cell gene DNA.

Cell Based Assays

The present invention provides a whole cell method to detect compound modulation of either KSHV modulated endothelial cell gene expression or the gene product. The method comprises the steps:

1) contacting a test compound with a cell that contains functional protein encoded by a KSHV modulated endothelial cell gene, or with an endothelial cell or cell line, and

2) measuring a change in the cell in response to modified KSHV modulated endothelial cell gene or gene product function by the test compound.

The amount of time necessary for cellular contact with the test compound is empirically determined, for example, by running a time course with a known modulator of the activity of the KSHV modulated endothelial cell gene or its gene product, and measuring cellular changes as a function of time.

The measurement means of the method of the present invention can be further defined by comparing a cell that has been exposed to a test compound to an identical cell that has not been similarly expose to the test compound. Alternatively two cells, one containing functional KSHV modulated endothelial cell gene and a second cell identical to the first, but lacking functional KSHV modulated endothelial cell gene could both be contacted with the same compound and compared for differences between the two cells. This technique is also useful in establishing the background noise of these assays. One of average skill in the art will appreciate that these control mechanisms also allow easy selection of cellular changes that are responsive to modulation of functional KSHV modulated endothelial cell gene or its gene product.

The term “cell” refers to at least one cell, but includes a plurality of cells appropriate for the sensitivity of the detection method. Cells suitable for the present invention may be bacterial, yeast, or eukaryotic.

The assay methods to determine compound modulation of functional KSHV modulated endothelial cell genes or gene products can be in conventional laboratory format or adapted for high throughput. The term “high throughput” refers to an assay design that allows easy analysis of multiple samples simultaneously, and capacity for robotic manipulation. Another desired feature of high throughput assays is an assay design that is optimized to reduce reagent usage, or minimize the number of manipulations in order to achieve the analysis desired. Examples of assay formats include 96-well or 384-well plates, levitating droplets, and “lab on a chip” micro-channel chips used for liquid handling experiments. It is well known by those in the art that as miniaturization of plastic molds and liquid handling devices are advanced, or as improved assay devices are designed, that greater numbers of samples may be performed using the design of the present invention.

The cellular changes suitable for the method of the present invention comprise directly measuring changes in the function or quantity of KSHV modulated endothelial cell genes or gene products, or by measuring downstream effects of KSHV modulated endothelial cell gene function, for example by measuring secondary messenger concentrations or changes in transcription or by changes in protein levels of genes that are transcriptionally influenced by KSHV modulated endothelial cell genes, or by measuring phenotypic changes in the cell. Preferred measurement means include changes in the quantity of KSHV modulated endothelial cell genes or the encoded protein, changes in the functional activity of KSHV modulated endothelial cell genes ot the encoded proteins, changes in the quantity of mRNA, changes in intracellular protein, changes in cell surface protein, or secreted protein, or changes in Ca+2, cAMP or GTP concentration. Changes in the levels of mRNA are detected by reverse transcription polymerase chain reaction (RT-PCR) or by differential gene expression. Immunoaffinity, ligand affinity, or enzymatic measurement quantifies changes in levels of protein in host cells. Protein-specific affinity beads or specific antibodies are used to isolate for example ³⁵S-methionine labelled or unlabelled protein. Labelled protein is analyzed by SDS-PAGE. Unlabelled protein is detected by Western blotting, cell surface detection by fluorescent cell sorting, cell image analysis, ELISA or RIA employing specific antibodies. Where the protein is an enzyme, the induction of protein is monitored by cleavage of a fluoro-genic or calorimetric substrate.

Preferred detection means for cell surface protein include flow cytometry or statistical cell imaging. In both techniques the protein of interest is localized at the cell surface, labeled with a specific fluorescent probe, and detected via the degree of cellular fluorescence. In flow cytometry, the cells are analyzed in a solution, whereas in cellular imaging techniques, a field of cells is compared for relative fluorescence.

A preferred detection means for secreted proteins that are enzymes such as alkaline phosphatase or proteases, would be fluorescent or calorimetric enzymatic assays. Fluorescent/luminescent/color substrates for alkaline phosphatase are commercially available and such assays are easily adaptable to high throughput multi-well plate screen format. Fluorescent energy transfer based assays are used for protease assays. Fluoro-phore and quencher molecules are incorporated into the two ends of the peptide substrate of the protease. Upon cleavage of the specific substrate, separation of the fluoro-phore and quencher allows the fluorescence to be detectable. When the secreted protein could be measure by radioactive methods, scintillation proximity technology could be used. The substrate of the protein of interest is immobilized either by coating or incorporation on a solid support that contains a fluorescent material. A radioactive molecule, brought in close proximity to the solid phase by enzyme reaction, causes the fluorescent material to become excited and emit visible light. Emission of visible light forms the basis of detection of successful ligand/target interaction, and is measured by an appropriate monitoring device. An example of a scintillation proximity assay is disclosed in U.S. Pat. No. 4,568,649, issued Feb. 4, 1986. Materials for these types of assays are commercially available from Dupont NEN® (Boston, Mass.) under the trade name FlashPlate™.

A preferred detection means where the endogenous gene results in phenotypic cellular structural changes is statistical image analysis the cellular morphology or intracellular phenotypic changes. For example, but not by way of limitation, and cell may change morphology such a rounding versus remaining flat against a surface, or may become growth-surface independent and thus resemble transformed cell phenotype well known in the art of tumor cell biology, or a cell may produce new outgrowths. Phenotypic changes that may occur intracellularly include cytoskeletal changes, alteration in the entoplasmic reticulum/Golgi complex in response to new gene transcription, or production of new vesicles.

Where the endogenous gene encodes a soluble intracellular protein, changes in the endogenous gene may be measured by changes of the specific protein contained within the cell lysate. The soluble protein may be measured by the methods described herein.

The present invention is also directed to methods for screening for compounds that modulate the expression of KSHV modulated endothelial cell genes as well as the function of the encoded protein in vivo. Compounds may modulate by increasing or attenuating the expression of DNA or RNA encoding the protein, or the function of the protein. Compounds that modulate the expression of KSHV modulated endothelial cell genes or the function of the encoded protein may be detected by a variety of assays. The assay may be a simple “yes/no” assay to determine whether there is a change in expression or function. The assay may be made quantitative by comparing the expression or function of a test sample with the levels of expression or function in a standard sample. Modulators identified in this process are useful as therapeutic agents.

Protein Based Assay

The present invention provides an in vitro protein assay method to detect test compound modulation of KSHV modulated endothelial cell gene encoded protein activity. The method comprises the steps:

1) contacting a test-compound, and functional KSHV modulated endothelial cell gene encoded protein, and

2) measuring a change to the protein function by the test compound.

The amount of time necessary for cellular contact with the compound is empirically determined, for example, by running a time course with a known protein modulator and measuring changes as a function of time.

Methods for detecting compounds that modulate proteolytic activity comprise combining a punitive modulating compound, functional protein, and a suitable labeled substrate and monitoring an effect of the compound on the protease by changes in the amount of substrate either as a function of time or after a predefined period of time. Labeled substrates include, but are not limited to; substrate that is radiolabeled (Coolican et al. (1986)J. Biol. Chem. 261:4170-6), fluorometric (Lonergan et al. (1995). J. Food Sci. 60:72-3, 78; Twining (1984). Anal. Biochem. 143:30-4) or colorimetric (Buroker-Kilgore and Wang (1993). Anal. Biochem. 208:387-92). Radioisotopes useful for use in the present invention include those well known in the art, specifically ¹²⁵I, ¹³¹I, ³H, ¹⁴C, ³⁵S, ³²P, and ³³P. Radioisotopes are introduced into the peptide by conventional means, such as iodination of a tyrosine residue, phosphorylation of a serine or threonine residue, or incorporation of tritium, carbon or sulfur utilizing radioactive amino acid precursors. Zymography following SDS polyacrylamide gel electrophoresis (Wadstroem and Smyth (1973). Sci. Tools 20:17-21), as well as by fluorescent resonance energy transfer (FRET)-based methods (Ng and Auld (1989). Anal. Biochem. 183:50-6) are also methods used to detect compounds that modulate proteolytic activity. Compounds that are agonists will increase the rate of substrate degradation and will result in less remaining substrate as a function of time. Compounds that are antagonists will decrease the rate of substrate degradation and will result in greater remaining substrate as a function of time.

A preferred assay format useful for the method of the present invention is a FRET based method using peptide substrates that contain a fluorescent donor with either a quencher or acceptor that are separated by a peptide sequence encoding the protease cleavage site. A fluorescent donor is a fluoro-genic compound that can adsorb energy and transfers a portion of the energy to another compound. Examples of fluorescent donors suitable for use in the present invention include, but are not limited to, coumarins, xanthene dyes such as fluoresceins, rhodols, and rhodamines, resorufins, cyanine dyes bimanes, acridines, isoindols, dansyl-dyes, aminophthalic hydrazides such as luminol and isoluminol derivatices, aminophthalimides, aminonapthalimides, aminobenzofurans, aminoquinolines, dicanohydroquinones, and europium and terbium complexes and related compounds.

A quencher is a compound that reduces the emission from the fluorescent donor when it is appropriately proximally located to the donor, and do not generally re-emit the energy in the form of fluorescence. Examples of such moieties include indigos, bezoquinones, anthraquinones, azo compounds, nitro compounds, indoanilines, and di- and triphenylmethanes. A FRET method using a donor/quencher pair measures increased emission from the fluorescent donor as a function of enzymatic activity upon the peptide substrate. Therefore a test compound that antagonizes KSHV modulated endothelial cell gene encoded protein will generate an emission signal between two control samples—a low (basal) fluorescence from the FRET peptide alone and a higher fluorescence from the FRET peptide digested by the activity of enzymatically active protein.

An acceptor is a fluorescent molecule that adsorbs energy from the fluorescent donor and re-emits a portion of the energy as fluorescence. An acceptor is a specific type of quencher that enables a separate mechanism to measure proteolytic efficacy. Methods that utilize a donor/acceptor pair measure a decrease in acceptor emission as a function of enzymatic activity upon the peptide substrate. Therefore a test compound that antagonizes KSHV modulated endothelial cell gene encoded protein will generate an emission signal between two control samples—a higher basal fluorescence from the FRET peptide alone and a lower fluorescence from the FRET peptide digested by the activity of enzymatically active protein. Examples of acceptor useful for methods of the present invention include, but are not limited to, coumarins, fluoresceins, rhodols, rhodamines, resorufins, cyanines, difuoroboradiazindacenes, and phthalcyanines.

Production and Use of Antibodies that Bind to KSHV Modulated Endothelial Cell Gene Encoded Proteins

Monospecific antibodies to KSHV modulated endothelial cell gene encoded proteins are purified from mammalian antisera containing antibodies reactive against the protein or are prepared as monoclonal antibodies reactive with the protein using the technique originally described by Kohler and Milstein, Nature 256: 495-497 (1975). Immunological techniques are well known in the art and described in, for example, Antibodies: A laboratory manual published by Cold Spring Harbor Laboratory Press, Cold Spring Harbor, N.Y., ISBN 0879693142. Monospecific antibody as used herein is defined as a single antibody species or multiple antibody species with homogenous binding characteristics for a protein. Homogenous binding as used herein refers to the ability of the antibody species to bind to a specific antigen or epitope, such as those associated with the KSHV modulated endothelial cell gene encoded protein, as described above. Protein specific antibodies are raised by immunizing animals such as mice, rats, guinea pigs, rabbits, goats, horses and the like, with rabbits being preferred, with an appropriate concentration of protein either with or without an immune adjuvant.

Preimmune serum is collected prior to the first immunization. Each animal receives between about 0.001 mg and about 1000 mg of protein associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of protein in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three-week intervals until maximal titers are obtained. At about 7 days after each booster immunization or about weekly after a single immunization, the animals are bled, the serum collected, and aliquots are stored at about −20° C.

Monoclonal antibodies (mAb) reactive with KSHV modulated endothelial cell gene encoded protein are prepared by immunizing inbred mice, preferably Balb/c, with the protein. The mice are immunized by the IP or SC route with about 0.001 mg to about 1.0 mg, preferably about 0.1 mg, of protein antigen in about 0.1 ml buffer or saline incorporated in an equal volume of an acceptable adjuvant, as discussed above. Freund's adjuvant is preferred, with Freund's complete adjuvant being used for the initial immunization and Freund's incomplete adjuvant used thereafter. The mice receive an initial immunization on day 0 and are rested for about 2 to about 30 weeks. Immunized mice are given one or more booster immunizations of about 0.001 to about 1.0 mg of protein antigen in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions that will allow the formation of stable hybridomas. Fusion partners may include, but are not limited to: mouse myelomas P3/NS1/Ag 4-1; MPC-1; S-194 and Sp2/0, with Sp2/0 being generally preferred. The antibody producing cells and myeloma cells are fused in polyethylene glycol, about 1000 mol. wt., at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using KSHV modulated endothelial cell gene encoded protein as the antigen. The culture fluids are also tested in the Ouchterlony precipitation assay to determine the isotype of the mAb. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973 or by the technique of limited dilution.

Monoclonal antibodies are produced in vivo by injection of pristane primed Balb/c mice, approximately 0.5 ml per mouse, with about 1×10⁶ to about 6×10⁶ hybridoma cells at least about 4 days after priming. Ascites fluid is collected at approximately 8-12 days after cell transfer and the monoclonal antibodies are purified by techniques known in the art.

In vitro production of mAb is carried out by growing the hybridoma in tissue culture media well known in the art. High density in vitro cell culture may be conducted to produce large quantities of mAbs using hollow fiber culture techniques, air lift reactors, roller bottle, or spinner flasks culture techniques well known in the art. The mAb are purified by techniques known in the art.

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar assays are used to detect the presence of KSHV modulated endothelial cell gene encoded protein in body fluids or tissue and cell extracts.

It is readily apparent to those skilled in the art that the above described methods for producing monospecific antibodies may be utilized to produce antibodies specific for polypeptide fragments, or full-length nascent polypeptide, or the individual subunits. Specifically, it is readily apparent to those skilled in the art that monospecific antibodies may be generated which are specific for only one protein subunit or the fully functional protein. It is also apparent to those skilled in the art that monospecific antibodies may be generated that inhibit normal function of KSHV modulated endothelial cell gene encoded protein.

Antibody affinity columns are made by adding the antibodies to a gel support such that the antibodies form covalent linkages with the gel bead support. Preferred covalent linkages are made through amine, aldehyde, or sulfhydryl residues contained on the antibody. Methods to generate aldehydes or free sulfhydryl groups on antibodies are well known in the art; amine groups are reactive with, for example, N-hydroxysuccinimide esters.

Kit Compositions Containing KSHV Modulated Endothelial Cell Gene or Gene Product Specific Reagents

Kits containing KSHV modulated endothelial cell genes DNA or RNA, antibodies to KSHV modulated endothelial cell gene encoded protein, or the KSHV modulated endothelial cell gene encoded protein may be prepared. Such kits are used to detect DNA which hybridizes to KSHV modulated endothelial cell gene DNA or to detect the presence of KSHV modulated endothelial cell gene encoded protein or peptide fragments in a sample. Such characterization is useful for a variety of purposes including but not limited to forensic analyses, diagnostic applications, and epidemiological studies.

The DNA molecules, RNA molecules, recombinant protein and antibodies of the present invention may be used to screen and measure levels of KSHV modulated endothelial cell gene DNA, KSHV modulated endothelial cell gene RNA or KSHV modulated endothelial cell gene encoded protein. The recombinant proteins, DNA molecules, RNA molecules and antibodies lend themselves to the formulation of kits suitable for a variety of uses, including but not limited to, the detection and typing of KSHV infected cells, Kaposi's sarcoma tissue, or tumor cells. Such a kit would comprise a compartmentalized carrier suitable to hold in close confinement at least one container. The carrier would further comprise reagents such as recombinant KSHV modulated endothelial cell gene encoded protein or anti-protein antibodies suitable for detecting KSHV modulated endothelial cell genes or gene products. The carrier may also contain a means for detection such as labeled antigen or enzyme substrates or the like.

Gene Therapy

Nucleotide sequences that are complementary to the KSHV modulated endothelial cell gene encoding DNA sequence can be synthesized for antisense therapy. These antisense molecules may be DNA, stable derivatives of DNA such as phosphorothioates or methylphosphonates, RNA, stable derivatives of RNA such as 2′-O-alkylRNA, or other KSHV modulated endothelial cell gene antisense oligonucleotide mimetics. KSHV modulated endothelial cell gene antisense molecules may be introduced into cells by microinjection, liposome encapsulation or by expression from vectors harboring the antisense sequence. KSHV modulated endothelial cell gene antisense therapy may be particularly useful for the treatment of diseases where it is beneficial to reduce KSHV modulated endothelial cell genes or the gene products' activity.

KSHV modulated endothelial cell gene gene therapy may be used to introduce KSHV modulated endothelial cell genes into the cells of target organisms. The KSHV modulated endothelial cell genes can be ligated into viral vectors that mediate transfer of the KSHV modulated endothelial cell gene DNA by infection of recipient host cells. Suitable viral vectors include retrovirus, adenovirus, adeno-associated virus, herpes virus, vaccinia virus, polio virus and the like. Alternatively, KSHV modulated endothelial cell gene DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo KSHV modulated endothelial cell genes gene therapy. KSHV modulated endothelial cell gene gene therapy may be particularly useful for the treatment of diseases where it is beneficial to elevate KSHV modulated endothelial cell genes activity. Protocols for molecular methodology of gene therapy suitable for use with the KSHV modulated endothelial cell genes is described in Gene Therapy Protocols, edited by Paul D. Robbins, Human press, Totawa N.J., 1996.

Pharmaceutical Compositions

Pharmaceutically useful compositions comprising KSHV modulated endothelial cell gene DNA, KSHV modulated endothelial cell gene RNA, or KSHV modulated endothelial cell gene encoded protein, or modulators of KSHV modulated endothelial cell genes and/or gene products activity, may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, or modulator.

Therapeutic or diagnostic compositions of the invention are administered to an individual in amounts sufficient to treat or diagnose disorders in which modulation of KSHV modulated endothelial cell gene or gene product-related activity is indicated. The effective amount may vary according to a variety of factors such as the individual's condition, weight, sex and age. Other factors include the mode of administration. The pharmaceutical compositions may be provided to the individual by a variety of routes such as subcutaneous, topical, oral and intramuscular.

The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Compounds identified according to the methods disclosed herein may be used alone at appropriate dosages defined by routine testing in order to obtain optimal inhibition of the KSHV modulated endothelial cell gene, gene product or its activity while minimizing any potential toxicity. In addition, co-administration or sequential administration of other agents may be desirable. For example, compounds that are useful for the treatment of Kaposi's sarcoma, such as daunorubicin, doxorubicin, interferon alpha, retinoids, and taxol, may be used in combination with a modulator of KSHV modulated endothelial cell genes or their gene products described herein. In addition, other known antitumor agents can be combined with the modulators of KSHV modulated endothelial cell genes or their gene products described herein.

The present invention also has the objective of providing suitable topical, oral, systemic and parenteral pharmaceutical formulations for use in the novel methods of treatment of the present invention. The compositions containing compounds or modulators identified according to this invention as the active ingredient for use in the modulation of KSHV modulated endothelial cell genes can be administered in a wide variety of therapeutic dosage forms in conventional vehicles for administration. For example, the compounds or modulators can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a KSHV modulated endothelial cell gene or gene product modulating agent.

The daily dosage of the products may be varied over a wide range from 0.01 to 1,000 mg per patient, per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. The dosages of the compounds or modulators are adjusted when combined to achieve desired effects. On the other hand, dosages of these various agents may be independently optimized and combined to achieve a synergistic result wherein the pathology is reduced more than it would be if either agent were used alone.

Advantageously, compounds or modulators of the present invention may be administered in a single daily dose, or the total daily dosage may be administered in divided doses of two, three or four times daily. Furthermore, compounds or modulators for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

For combination treatment with more than one active agent, where the active agents are in separate dosage formulations, the active agents can be administered concurrently, or they each can be administered at separately staggered times.

The dosage regimen utilizing the compounds or modulators of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

In the methods of the present invention, the compounds or modulators herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

For liquid forms the active drug component can be combined in suitably flavored suspending or dispersing agents such as the synthetic and natural gums, for example, tragacanth, acacia, methyl-cellulose and the like. Other dispersing agents that may be employed include glycerin and the like. For parenteral administration, sterile suspensions and solutions are desired. Isotonic preparations, which generally contain suitable preservatives, are employed when intravenous administration is desired.

Topical preparations containing the active drug component can be admixed with a variety of carrier materials well known in the art, such as, eg., alcohols, aloe vera gel, allantoin, glycerine, vitamin A and E oils, mineral oil, PPG2 myristyl propionate, and the like, to form, eg., alcoholic solutions, topical cleansers, cleansing creams, skin gels, skin lotions, and shampoos in cream or gel formulations.

The compounds or modulators of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds or modulators of the present invention may also be coupled with soluble polymers as targetable drug carriers. Such polymers can include polyvinyl-pyrrolidone, pyran copolymer, polyhydroxypropylmethacryl-amidephenol, polyhydroxy-ethylasparta-midephenol or polyethyl-eneoxidepolylysine substituted with palmitoyl residues. Furthermore, the compounds or modulators of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

For oral administration, the compounds or modulators may be administered in capsule, tablet, or bolus form or alternatively they can be mixed in the animals feed. The capsules, tablets, and boluses are comprised of the active ingredient in combination with an appropriate carrier vehicle such as starch, talc, magnesium stearate, or di-calcium phosphate. These unit dosage forms are prepared by intimately mixing the active ingredient with suitable finely-powdered inert ingredients including diluents, fillers, disintegrating agents, and/or binders such that a uniform mixture is obtained. An inert ingredient is one that will not react with the compounds or modulators and which is non-toxic to the animal being treated. Suitable inert ingredients include starch, lactose, talc, magnesium stearate, vegetable gums and oils, and the like. These formulations may contain a widely variable amount of the active and inactive ingredients depending on numerous factors such as the size and type of the animal species to be treated and the type and severity of the infection. The active ingredient may also be administered as an additive to the feed by simply mixing the compound with the feedstuff or by applying the compound to the surface of the feed. Alternatively the active ingredient may be mixed with an inert carrier and the resulting composition may then either be mixed with the feed or fed directly to the animal. Suitable inert carriers include corn meal, citrus meal, fermentation residues, soya grits, dried grains and the like. The active ingredients are intimately mixed with these inert carriers by grinding, stirring, milling, or tumbling such that the final composition contains from 0.001 to 5% by weight of the active ingredient.

The compounds or modulators may alternatively be administered parenterally via injection of a formulation consisting of the active ingredient dissolved in an inert liquid carrier. Injection may be either intramuscular, intraruminal, intratracheal, or subcutaneous. The injectable formulation consists of the active ingredient mixed with an appropriate inert liquid carrier. Acceptable liquid carriers include the vegetable oils such as peanut oil, cottonseed oil, sesame oil and the like as well as organic solvents such as solketal, glycerol formal and the like. As an alternative, aqueous parenteral formulations may also be used. The vegetable oils are the preferred liquid carriers. The formulations are prepared by dissolving or suspending the active ingredient in the liquid carrier such that the final formulation contains from 0.005 to 10% by weight of the active ingredient.

Topical application of the compounds or modulators is possible through the use of a liquid drench or a shampoo containing the instant compounds or modulators as an aqueous solution or suspension. These formulations generally contain a suspending agent such as bentonite and normally will also contain an antifoaming agent. Formulations containing from 0.005 to 10% by weight of the active ingredient are acceptable. Preferred formulations are those containing from 0.01 to 5% by weight of the instant compounds or modulators.

This invention also relates to methods of doing business comprising the steps of determining the level of RNA expression for an RNA sample, wherein the RNA sample is amplified and fluorescently labeled, hybridizing the fluorescently labeled RNA to a microarray containing a plurality of nucleic acid sequences representing a gene expression profile for a particular cell or tissue type (e.g., neuronal cell or disease tissue), and scanning the microarray for fluorescence detection; normalizing said fluorescence; and using a signature extraction algorithm (e.g., MaxCor or Mean Log algorithms) to create a gene expression profile. In a preferred embodiment, the RNA sample is obtained from a patient and the patient sample includes, but is not limited to, blood, amniotic fluid, plasma, semen, bone marrow, and tissue biopsy.

The present invention also relates to business methods where gene expression profiles are used for analyzing test samples (e.g., patient samples). In a specific embodiment, this method may be accomplished using the gene expression profile microarrays of the present invention. For example, a user (e.g., a health practitioner such as a physician) may obtain a sample (e.g., blood, tissue biopsy) from a patient. The sample may be prepared in-house, for example, using hospital facilities or the sample may be sent to a commercial laboratory facility. Briefly, RNA is extracted from the patient sample using methods that are well-known in the art (Sambrook, et al. (1989)). The RNA is, for example, then amplified by PCR, labeled with a fluorophore, and hybridized to a support representing a particular gene expression profile. The support is scanned for fluorescence and the results of the scan may be sent to a central gene expression profile database for analysis. In another embodiment, the sample itself is sent to a central laboratory facility for scanning analysis. The scanning results may be sent to the central laboratory facility for analysis via a computer terminal and through the Internet or other means. The connection between the user and the computer system is preferably secure. In practice, the user may input, for example, information relating to the fluorescence scanning results of the support as well as additional information concerning the patient such as the patient's disease state, clinical chemistry (e.g., red blood cell count, electrolytes), and other factors relating to the patient's disease state. The central computer system may then, through the use of resident computer programs, provide an analysis of the patient's sample and generate a gene expression profile reflecting the patient's genetic profile.

Those skilled in the art will appreciate that the methods and apparatus of the present invention apply to any computer system, regardless of whether the computer system is a complicated multi-user computing apparatus or a single user device such as a personal computer or workstation. Computer system suitably comprises a processor, main memory, a memory controller, an auxiliary storage interface, and a terminal interface, all of which are interconnected. Note that various modifications, additions, or deletions may be made to the computer system within the scope of the present invention such as the addition of cache memory or other peripheral devices.

The processor performs computation and control functions of the computer system, and comprises a suitable central processing unit (CPU). The processor may comprise a single integrated circuit, such as a microprocessor, or may comprise any suitable number of integrated circuit devices and/or circuit boards working in cooperation to accomplish the functions of a processor.

In a preferred embodiment, the auxiliary storage interface allows the computer system to store and retrieve information from auxiliary storage devices, such as magnetic disk (e.g., hard disks or floppy diskettes) or optical storage devices (e.g., CD-ROM). One suitable storage device is a direct access storage device (DASD). A DASD may be a floppy disk drive which may read programs and data from a floppy disk. It is important to note that while the present invention has been (and will continue to be) described in the context of a fully functional computer system, those skilled in the art will appreciate that the mechanisms of the present invention are capable of being distributed as a program product in a variety of forms, and that the present invention applies equally regardless of the particular type of signal bearing media to actually carry out the distribution. Examples of signal bearing media include: recordable type media such as floppy disks and CD ROMS, and transmission type media such as digital and analog communication links, including wireless communication links.

The computer systems may also comprise a- memory controller, through use of a separate processor, which is responsible for moving requested information from the main memory and/or through the auxiliary storage interface to the main processor. While for the purposes of explanation, the memory controller is described as a separate entity, those skilled in the art understand that, in practice, portions of the function provided by the memory controller may actually reside in the circuitry associated with the main processor, main memory, and/or the auxiliary storage interface.

Furthermore, the computer systems may comprise a terminal interface that allows system administrators and computer programmers to communicate with the computer system, normally through programmable workstations. It should be understood that the present invention applies equally to computer systems having multiple processors and multiple system buses. Similarly, although the system bus of the preferred embodiment is a typical hardwired, multidrop bus, any connection means that supports bidirectional communication in a computer-related environment could be used.

The main memory of the computer systems suitably contains one or more computer programs relating to the algorithms used to generate the gene expression profiles and an operating system. Computer program in memory is used in its broadest sense, and includes any and all forms of computer programs, including source code, intermediate code, machine code, and any other representation of a computer program. The term “memory” as used herein refers to any storage location in the virtual memory space of the system. It should be understood that portions of the computer program and operating system may be loaded into an instruction cache for the main processor to execute, while other files may well be stored on magnetic or optical disk storage devices. In addition, it is to be understood that the main memory may comprise disparate memory locations.

Another preferred embodiment of the present invention comprises a variety of business methods including methods for screening drug and toxicity effects on tissue or cell samples, as well as methods for the discovery of new drugs to treat disease. A further embodiment of the present invention comprises a business method of providing gene expression profiles for normal and disease tissues. Also within the scope of this invention are business methods providing diagnostics and predictors for patient samples.

The business methods of the present application relate to the commercial and other uses, of the methodologies of the present invention. In one aspect, the business methods include the marketing, sale, or licensing of the present methodologies in the context of providing consumers, i.e., patients, medical practitioners, medical service providers, and pharmaceutical distributors and manufacturers, with the gene expression profiles provided by the present invention.

The gene expression profile database may be an internal database designed to include annotation information about the gene expression profiles generated by the methods of the present invention. Such information may include, for example, the microarray in which a given nucleic acid sequence was found, descriptive information about a related cDNAs associated with the sequence, tissue or cell source, sequence data obtained from external sources, and preparation methods. The database may divide into two sections: one for storing the sequences and the other for storing the associated information. This database may be maintained as a private database with a fire-wall within the central computer facility. However, this invention is not so limited and the gene expression profile database may be made available to the public.

The database may be a network system connecting the network server with clients. The network may be any one of a number of conventional network systems, including a local area network (LAN) or a wide area network (WAN), as is known in the art (e.g., Ethernet). The server may include software to access database information for processing user requests, and to provide an interface for serving information to client machines. The server may support the World Wide Web and maintain a web-site and Web browser for client use. Client/server environments, database servers, and networks are well-documented in the technical, trade, and patent literature.

Through the Web browser, clients may construct search requests for retrieving data from a microarray database and a gene expression database. For example, the user may “point and click” to user interface elements such as buttons, pull down menus, and scroll bars. The client requests may be transmitted to a Web application which formats them to produce a query that may be used to gather information from the microarray database or gene expression database. In addition, the web-site may provide hypertext links to public databases such as GenBank and associated databases maintained by the National Center for Biotechnology Information (NCBI), part of the National Library of Medicine as well as, any links providing relevant information for gene expression analysis, genetic disorders, scientific literature, and the like.

The following examples illustrate the present invention without, however, limiting the same thereto.

### EXAMPLES

### Example 1

Derivation of KSHV-Infected DMVEC

Establishment of KSHV-infected DMVEC has been described previously in detail⁽⁴³⁾. Briefly, dermal microvascular cells immortalized by retroviral expression of the E6 and E7 genes of human papillomavirus type 16 (DMVEC) were infected with KSHV derived from the supernatant of tetradecanoyl phorbol acetate (TPA)-stimulated BCBL-1 cells. Infection was verified by DNA PCR for amplification of the KS330 BamHI fragment of the ORF 26 gene, RT-PCR for the spliced mRNA from the ORF29 gene⁽¹¹³⁾. The percentage of latently infected cells was determined by immunofluorescent staining for LANA/ORF73⁽⁸²⁾. Lytic induction was evaluated with antibodies against an early lytic protein ORF59/PF-8⁽¹¹⁰⁾ and a late lytic glycoprotein protein ORF K8.1A/B⁽¹¹¹⁾. DMVEC were used for experiments when >90% of cells expressed ORF73. In the absence of chemical induction, 2-5% of infected cells expressed ORF59 with approximately 10% of ORF59-positive cells expressing K8.1A/B. Antibodies against viral proteins were a generous gift from Dr. Bala Chandran.

DNA Microarrays

DNA microarrays were prepared as described in Salunia et al. (57) by spotting PCR fragments of human cDNAs onto glass slides (Molecular Dynamics) using a microarrayer (Molecular Dynamics). One DNA microarrays was used: Mega-A chip: 4428 clones. Each clone was spotted in duplicate. Each microarray contained 30 plant genes as background controls⁽⁶⁰⁾. Hybridization signals were scanned and normalized as described⁽⁵⁷⁾.

Probe Preparation and Hybridization

Transformed dermal microvascular endothelial cells infected with KSHV were propagated as previously described⁽⁴³⁾. Two different uninfected and three different infected cell lines were either not induced or induced with PMA for 6 hours or 48 hours and harvested by trypsinization. Total RNA was prepared using RNeasy columns (Qiagen), after which each sample was DNase (Promega) treated and re-purified on RNeasy columns. Messenger RNA was amplified once using a T7 polymerase linear amplification protocol⁽⁵⁷⁾. After amplification, the RNA was labeled by reverse transcription in the presence of Cy3-Cytidine.

Corresponding infected and non-infected samples were hybridized as described⁽⁵⁷⁾. Each labeled and amplified RNA was hybridized to at least two separate chips to enable a coefficient of variation (CV) determination, and the average intensity for genes with a CV below 50% was used for subsequent calculation of expression ratios. Results for each microarray were normalized to the 75^{th} percentile as described⁽⁵⁷⁾.

RT-PCR for SCF and c-Kit

Total RNA was isolated from mock and KSHV-infected DMVEC using an RNeasy Total RNA kit (Qiagen, Chatsworth, Calif.) according to the manufacturer's instructions. RNA was quantified by spectrophotometry and cDNA was synthesized using superscript reverse transcriptase (RT; Life Technologies, Gaithersburg, Md.) at 200U/1 μg of RNA. The primers used for amplification of c-Kit and SCF mRNA were: c-Kit, 5′-CTCAACCATCTGTGAGTCCA-3′ (SEQ ID NO: 1) and 5′-AAGCCGTGTTTGTTGGTG CA-3′ (SEQ ID NO: 2)⁽⁸³⁾, and SCF, 5′-CCATTGATGCCTTCAAGGAC-3′ (SEQ ID NO: 3) and 5′-CTTCCAGTATAAGGCTCCAA-3′ (SEQ ID NO: 4)⁽⁸⁴⁾, which yielded products of 242 bp and 274 bp for c-Kit and SCF respectively. The cellular gene hypoxanthine-guanine phosphoribosyltransferase (HPRT) was amplified from each sample as a control for cDNA synthesis. Consistent HPRT amplificates were obtained from samples under comparison. For each reaction, samples in the absence of RT were included. PCR products were resolved by agarose gel electrophoresis and visualized by ethidium bromide staining.

Immunofluorescent Staining

For detection of c-Kit protein, DMVEC monolayers were rinsed in PBS containing 1% bovine serum albumen (BSA) and 0.02% sodium azide (staining buffer), and stained with anti-c-Kit monoclonal antibodies, clone Nu-c-kit (Research Diagnostics, Flanders, N.J.) or Clone 57A5 (Ancell, Bayport, Minn.) followed by a goat anti-mouse FITC-conjugated antibody (Biosource International, Camarillo, Calif.). Both antibodies were used at a 1:100 dilution in staining buffer for 60 minutes at 37° C. Stained cells were fixed in 2% paraformaldehyde, mounted and examined on a Nikon fluorescent microscope. Omission of primary antibody from duplicate monolayers revealed no non-specific binding of second conjugate. Immunofluorescent analysis was performed on mock and KSHV-infected DMVEC as well as DMVEC infected with adenovirus constructs expressing c-Kit proteins.

SCF ELISA

Supernatants were harvested from confluent monolayers of mock and KSHV-infected DMVEC after incubation periods ranging from 6 to 48 hours. SCF present in harvested supernatants was quantitated using an SCF ELISA detection kit (R&D) according to the manufacturers instructions.

Proliferation Assays

Proliferation of mock and KSHV-infected DMVEC, and the SCLC line HTB-172, was quantitated using an XTT-based assay. Cells were added to Primaria 96 well trays (Becton-Dickinson) at 1×104 or 5×104 cells/well. Exogenous SCF (20, 50 and 100 ng/ml) and STI 571 (0.01, 0.1, 1 and 10 μM) were added 24 hours after plating as designated by the experimental protocol. XTT (Roche, Molecular Biochemicals, Indianapolis, Ind.) was added 48 hours later according to the manufacturers instructions. Absorbance was read after 4 to 6 hours on a microplate reader. The HTB-172 line was obtained from the American Type Culture Collection (ATCC, Rockville, Md.).

Construction and Use of Adenovirus Vectors

A dominant-negative c-Kit mutant (c-Kit/DN) was constructed by insertion of a premature stop codon at Ser614 in the cytoplasmic domain using standard PCR-based mutagenesis. Truncation of c-Kit at this site deletes the ATP-binding and phosphotransferase domains without affecting the dimerization domain. Following DNA sequence analysis to confirm mutagenesis, c-Kit/DN was cloned into an adenoviral expression vector as previously described⁽¹¹⁵⁾. This places c-Kit/DN under the control of a tet-responsive promoter/enhancer element and protein expression is driven by coinfection with an adenovirus expressing the ‘tet-off’ trans-activator (Ad/trans). Recombinant viruses were screened by PCR, and protein expression was confirmed by western immunoblot of infected cell lysates using a rabbit polyclonal antibody directed against the N-terminus of c-Kit (H-300; Santa Cruz Biotechnology, Inc., Santa Cruz, Calif.). For over expression of c-Kit in DMVEC in the absence of KSHV, an adenovirus expressing wild-type c-Kit (c-Kit/WT) was constructed in an identical manner. All recombinant adenoviruses were plaque-purified and viral stocks were grown and titered on 293 cells. For DMVEC infection, monolayers were incubated with c-Kit/WT or c-Kit/DN at a multiplicity of infection (MOI) of 1:10 to 1:100 and Ad/trans at an MOI 1:10 for four hours at 37° C. Optimal MOI doses were previously determined. Virus stocks were diluted in medium containing 2% human serum and polybrene (4 μg/ml). As a control for infection efficiency and non-specific effect of adenovirus infection, duplicate monolayers were infected with an adenovirus vector expressing green fluorescent protein (Ad/GFP). Infection with Ad/GFP and Ad/trans at MOI of 1:100 and 1:10 respectively allowed infection of >80% of cells in culture with minimal cytopathic effect.

Transformation Assays

To promote a transformed phenotype, KSHV-infected DMVEC were cultured post-confluency in 35 mm Primaria culture dishes. Under these conditions, cells assumed a pronounced spindle morphology, exhibited a disorganized growth pattern and developed multilayered foci within the monolayer. Uninfected DMVEC cultured under similar conditions displayed growth inhibition and maintained a cobblestone phenotype with organized cell borders. Post-confluent cells were infected with adenovirus constructs as described above or exposed to STI 571 at increasing doses (0.01, 0.1, 1 and 10 μM). STI 571 was replenished every 48 to 36 hours using dilutions freshly prepared from a frozen stock. Cells were examined daily for evidence of phenotypic change using a phase-contrast microscope and results recorded photographically.

Reagents

The 2-phenylaminopyrimidine derivative STI 571 was developed and generously provided by Dr. Elisabeth Buchdunger (Novartis, Basel, Switzerland). Stock solutions of STI 571 were prepared at 10 mmol/L by dissolving 5 mg STI 571 in 1 ml PBS, and used fresh or stored at −20° C. Working solutions were diluted in Endothelial-SFM immediately prior to use.

PDTC (pyrrolidinedithiocarbamate), trans-retinoic acid, and SB203580 were purchase from Sigma Chemical Co. (St. Louis, Mo.). Calcitonin gene-related peptide (CGRP) and CGRP-8-37 peptide were purchased from Bachem. InCELLect AKAP St-Ht31 and St-Ht31-control peptide were purchased from Promega Corporation (Madison, Wis.). Haloperidol and phorbol-112-myristate-13-acetate (PMA) were purchased from Calbiochem (San Diego, Calif.). T22 was purchased American Peptides International, Inc. (Sunnyvale, Calif.). Ganciclovir, BQ788 was purchased from Peptides International, Inc. (Louisville, Ky.). 15-deoxy⁽¹²⁻¹⁴⁾ prostaglandin J2 was purchased from Cayman Chemicals (Ann Arbor, Mich.).

RT PCR

KSHV genes ORF 64 and K12 (kaposin) were used as reverse transcriptase PCR (RT-PCR) templates as described previously⁽⁴³⁾. ORF 65 sequences were amplified with primers 5-GGCGTTAATTAAGCTAGCATGTCCAACTTTAAGGTGAGA (SEQ ID NO: 5) and 5′-AAACCTATTTCTTTTTTGCCAGAGG (SEQ ID NO: 6). Cellular genes (HPRT, GAPDH, or -actin) were amplified from each sample as a control for cDNA synthesis. PCR products were visualized following electrophoresis on agarose-ethidium bromide gels.

Global RNA Changes in Cells Infected with KSHV

We have previously described an in vitro model based on KSHV infection of dermal microvascular endothelial cells (DMVEC) where the majority of virus-challenged cells harbor latent virus and up to 40% of cells can be chemically induced to undergo lytic replication⁽⁴³⁾. Due to the high percentage of cells harboring latent virus and the high efficiency of induction of lytic replication in this system, it was possible to directly isolate RNA from tissue culture cells without further enrichment of infected cells. RNA samples from three infected and two uninfected DMVEC lines were prepared from non-induced cells and cells induced with PMA for 6 and 48 hours. RNA of non-infected cells and infected cells was converted into cy-3 fluorescently labeled cDNA, respectively and hybridized to a DNA chips displaying a total of 4428 PCR fragments. Each chip contained two sets of the PCR fragments and hybridizations were performed in duplicate so that both intra- as well as interchip variation was controlled for. A comparison of two independent hybridizations is shown in FIG. 1c. Variations between chip to chip signals were less than 1.8 fold for 99.9% of the signals. Therefore ratios greater than 1.8 were considered significant. All hybridization signals obtained with the same clone were used to calculate the arithmetic mean individual clones were found to be induced more than 1.8 fold in at least 2 out of three experiments per time point, whereas other clones were repressed more than 1.8 fold (ratio<0.55) for at least one of the three time points analyzed. Ratios for all clones are shown in Table 2, If several clones represented the same gene, results for all individual clones are shown. Variations in signal ratios observed in these cases are caused by the fact that hybridization signals can differ for different segments of the same gene.

Genes Involved in Angiogenesis

Among the induced genes in KSHV-infected cells were several members of the extracellular matrix metalloproteinases (MMP) family (table 2). MMPs degrade components of the extracellular matrix (ECM). Various types of MMPs were up regulated as well as ECM degrading proteases. The present invention also, therefore, includes methods and compositions for the modulation of endothelial cell transformation, invasiveness of transformed endothelial cells, and neo-vascularization.

The data further shows that KSHV modulates the gene expression pattern of factors regulating the proliferation of endothelial cells. Induction of pro-angiogenic factors by KSHV could impose a growth advantage over uninfected cells, which might explain the observation that the percentage of KSHV-infected endothelial cells increases during progression of KS⁽²⁰⁾. The present invention is, therefore, also drawn to methods and compositions for the modulation of endothelial cell proliferation, including increasing or decreasing endothelial cell proliferation.

Interferon-Induced Genes

Another group of genes strongly induced in KSHV-infected cells is known to be induced by interferon (Table 2).

Inflammation

Several pro-inflammatory cytokines and several pro-inflammatory genes were upregulated by KSHV (Table 2). The strong induction of these pro-inflammatory and chemoattractant cytokines during the lytic phase of infection compared to the latent phase, suggests that KSHV-infected cells play a major role in the attraction of monocytes and leukocytes to KS tumors.

Differentiation Markers

KSHV modulated the expression pattern of several known differentiation markers as well as genes with known functions in cell differentiation (Table 2). Most prominently c-kit was increased above its normal expression level in endothelial cells. The KSHV-regulated differentiation markers are representative of different mesenchymal lineage originating from bone marrow stromal cells. Adipocytes, osteoblasts and chondrocytes are derived from multipotent mesenchymal stem cells⁽⁵⁴⁾ whereas ECs and hematopoietic cells differentiate from a common progenitor, the hemangioblast⁽¹⁸⁾. Expression of differentiation markers corresponding to these various lineages indicates that KSHV induces dedifferentiation of infected endothelial cells.

Expression of c-Kit in DMVEC is Up-Regulated by KSHV Infection

One of the most consistently upregulated genes in the DNA microarray analysis of KSHV-infected DMVEC was the c-Kit gene (Table 2). In agreement with microarray results, enhanced expression of c-Kit mRNA and surface protein on KSHV-infected DMVEC was shown by RT-PCR (FIG. 1A), and immunofluorescent staining (FIG. 1B), respectively. Earlier studies have shown that endothelial cells from umbilical vein and aorta co-express c-Kit and the SCF ligand^{(100, 101, 102, 103)}. To determine whether DMVEC expressed SCF in addition to c-Kit, we performed RT-PCR analysis for SCF mRNA. Although SCF mRNA was readily detected in both KSHV-infected and uninfected DMVEC, indicating co-expression of receptor and ligand in these cells, no virus-induced change in SCF expression levels was noted (FIG. 2A). Due to alternative RNA splicing, the SCF protein exists in both membrane-bound and soluble forms⁽¹⁰⁴⁾. The smaller PCR product depicted in FIG. 2A reflects the membrane bound form of the protein, suggesting that the ratio of membrane-bound to soluble SCF in DMVEC is also unaffected by KSHV infection. RT-PCR using isoform-specific primers to directly distinguish between membrane-bound and soluble SCF transcripts⁽¹⁰⁵⁾ confirmed this conclusion. Consistent with RT-PCR results, analysis of DMVEC culture supernatants using a SCF-specific ELISA to measure secretion of SCF showed no significant changes in protein levels associated with KSHV infection (FIG. 2B). Thus, if KSHV infection alters c-Kit/SCF regulated signaling pathways, the contribution of virus infection is presumably at the level of c-Kit expression.

c-Kit Expression in KSHV-Infected DMVEC Promotes Proliferation in Response to Exogenous SCF

To examine whether KSHV-induced up-regulation of c-Kit had functional consequences, we tested whether the mitogenic response of DMVEC to exogenous SCF was enhanced following KSHV infection. Mock- and KSHV-infected DMVEC were cultured in growth factor depleted medium in the absence and presence of recombinant SCF and proliferation was measured using an XTT-based dye reduction assay⁽¹¹²⁾. Both mock- and KSHV-infected DMVEC exhibited a dose-dependent proliferation in response to exogenous SCF that was maximal at 50 to 100 ng/ml. However, infected DMVEC were significantly more responsive to exogenous ligand than mock-infected DMVEC (FIG. 3A). In mock-infected cultures, the increase over basal proliferation in the absence of recombinant SCF was never more than 40%, while proliferation of KSHV-infected DMVEC was increased by as much as 85%. During KS disease, an enhanced capacity of KSHV-infected endothelial cells to respond to SCF produced by adjacent endothelial cells, or by macrophages and mast cells infiltrating the KS lesion, would promote more rapid growth of virus infected cells.

The c-Kit Tyrosine Kinase Inhibitor STI 571 Inhibits the Proliferation of KSHV-infected DMVEC

To confirm a role for c-Kit/SCF in the enhanced growth response of KSHV-infected DMVEC, proliferation of infected DMVEC in response to exogenous SCF was measured in the presence of increasing concentrations of a 2-phenylaminopyrimidine derivative, STI 571 (formally known as CGP 57148B)^{(106, 97)} an inhibitor of c-Kit tyrosine kinase activity⁽⁹⁹⁾. As illustrated in FIG. 3B, the proliferative response of KSHV-infected DMVEC to exogenous SCF was completely inhibited by a 1 μM dose of STI 571. Testing of DMVEC viability by trypan blue exclusion showed that growth inhibition was not due to non-specific cytotoxicity of STI 571. In addition, STI 571 had no effect on the capacity of the human Jurkat T cell line to proliferate in response to exogenous IL-2. The capacity of STI 571 to inhibit KSHV-infected DMVEC proliferation confirms a role for c-Kit signaling in the growth response of KSHV-infected cells and further suggests a novel strategy for KS therapy.

Expression of C-Kit in DMVEC is Sufficient for Induction of Morphological Transformation

While induction of growth signaling loops is the most widely described consequence of abnormal c-Kit/SCF activity in tumor cell lines, enhanced c-Kit expression has also been directly associated with changes in cell morphology and acquisition of a transformed phenotype. For example, ectopic expression of c-Kit in murine fibroblasts induces morphologic alteration, growth in soft agar and tumorigenicity in nude mice^{(107, 108, 109)}. We previously demonstrated that KSHV-infected DMVEC develop a spindle morphology and characteristics of transformation including disorganized growth, focus formation and anchorage-independent growth in semi-solid agar⁽⁴³⁾. To determine whether c-Kit up-regulation was sufficient to induce the morphologic changes caused by KSHV infection, c-Kit was over-expressed in normal DMVEC in the absence of any KSHV genes. The c-Kit over-expression was achieved by infecting DMVEC with an adenovirus vector expressing wild type c-Kit protein (Ad/c-KitWT) along with an adenovirus expressing a transactivator (Ad/trans) necessary for induction of c-Kit gene expression. To control for infection efficiency and non-specific effects of adenovirus infection, parallel DMVEC cultures were infected with an adenovirus expressing green fluorescent protein (Ad/GFP) and Ad/trans. GFP, as visualized by fluorescence microscopy, was expressed at high levels in the majority (>80%) of DMVEC. Immunofluorescent staining of DMVEC infected with Ad/c-KitWT at a comparable multiplicity of infection (MOI) showed strong surface expression of the c-Kit protein on approximately 50% of cells. As illustrated in FIG. 4A, c-Kit over-expression had a dose-dependent effect on DMVEC morphology that was identical to that observed following KSHV infection. Ad/c-KitWT-infected cells became spindle-shaped and disorganized, with overgrowth of the monolayer and a loss of discrete cell borders. Spindle formation was restricted to those cells that expressed high levels of c-Kit protein on the cell surface (FIG. 4B). Morphological changes were first noted at day 5 post-infection (PI), and were prominent by day 10 PI. The changes were specific to adenovirus-infected cultures over-expressing c-Kit, since DMVEC infected with Ad/GFP were maintained for up to three weeks without phenotypic alteration. Addition of exogenous SCF to Ad/GFP or Ad/c-KitWT-infected DMVEC did not induce or accentuate morphologic change, and adenovirus infection did not alter SCF production by DMVEC. These results show that the endogenous SCF produced by DMVEC is sufficient to activate the pathways leading to morphologic transformation. Importantly, these studies demonstrate that increased expression of c-Kit in DMVEC is sufficient to induce morphologic changes comparable to those observed following KSHV infection of DMVEC.

Inhibition of C-Kit Activity Reverses KSHV-Induced Transformation

To directly test whether KSHV-induction of c-Kit is necessary for virus-induced transformation, the consequence of inhibiting c-Kit signaling in KSHV-transformed DMVEC was evaluated. As illustrated in FIG. 5A, KSHV-transformed DMVEC exhibit disorganized growth, loss of contact inhibition and focus formation in monolayer culture. However, following treatment of DMVEC with STI 571 to inhibit endogenous c-Kit tyrosine kinase activity, focus formation was inhibited and an organized monolayer with distinct cell margins was re-established. The effect of STI 571 was dose-dependent and complete at a drug concentration of 1 μM. The loss of transformed growth characteristics was not due to drug-induced cytotoxicity since removal of STI 571 led to regeneration of the transformed phenotype, even after exposure of cells to a 10 μM dose. Uninfected DMVEC exhibited normal growth with an organized cobblestone phenotype when maintained at confluency, and exposure to STI 571 had no effect on cell morphology or viability.

Because STI 571 is also active against the Abl and platelet-derived growth factor (PDGF) β receptor tyrosine kinases^{(106, 98)}, the inhibitory activity in DMVEC could imply a role for one or other of these receptors in KSHV-induced transformation. DNA microarray analysis of DMVEC did not reveal any KSHV-induced up-regulation of Abl, PDGF or PDGF-receptor genes suggesting that c-Kit was the primary drug target. However, to confirm a central role for c-Kit in KSHV-induced DMVEC transformation, we designed a complementary approach to specifically inhibit c-Kit signaling in KSHV-transformed DMVEC. In this approach, a dominant negative c-Kit protein lacking the cytoplasmic ATP-binding and phosphotransferase domains necessary for c-Kit signaling (Ad/c-KitDN) was expressed in KSHV-infected cells using the adenovirus delivery system outlined above. In control cultures infected with Ad/GFP alone, no change in the transformed phenotype was observed. In contrast, expression of the dominant negative c-Kit protein in KSHV-transformed DMVEC resulted in a dramatic loss of transformed foci with cells flattening out and becoming organized in a manner identical to that observed following STI 571 treatment (FIG. 5B). The ability to reverse KSHV-induced morphological transformation through specific inhibition of c-Kit activity demonstrates a critical role for c-Kit signaling in KSHV-induced transformation of endothelial cells and supports a role for up-regulation of c-Kit as a factor in KS tumorigenesis.

Function of Host Gene Products Induced During Lytic Replication

The induction or repression of cellular pathways by viral infection is either a cellular defense mechanism, a directed modulation of host gene expression by some of the viral gene products, or a neutral bystander effect. One way to distinguish between these possibilities is by examining whether or not the inhibition or activation of a given cellular pathway affects viral replication.

Host genes specifically induced during lytic replication were identified by comparing hybridization intensities between KSHV-infected cells (I7) induced for 6 hours or 48 hours with corresponding non-induced KSHV-infected cells together with untreated and PMA-treated non-infected cells. Genes showing hybridization signals at least two-fold above the maximum hybridization signal obtained in any of the controls were selected for further analysis. The 23 genes that fulfilled these criteria are shown in Table 1. Genes up-regulated 6 and 48 hours after PMA induction of KSHV-infected cells targeted by various compounds. Direct target is the gene targeted by the compound, indirect target is the gene up-regulated on the chip if different from the gene targeted.

To evaluate the significance of these genes for viral replication, we selected compounds known to modulate either the induction of specific host genes or the function of the gene product. For most genes it was possible to identify specific antagonists. Several genes induced at 6 hours post-induction are known to be induced by pro-inflammatory stimuli. Well-known examples are A20, IL-8, ICAM-1 and cox-2. However, other genes in this group, such as jagged1, MMP-10 or angiopoietin-2 have also been shown to be induced by inflammatory signals. Since activation of NFkB is essential for this transcriptional response, we analyzed whether 1-pyrrolidinecarbodithioic acid (PDTC), an inhibitor of NFkB activation⁽⁶¹⁾, would modulate the expression of KSHV genes during lytic replication. Among other genes induced at 6 hours was the purine nucleoside phosphorylase (PNP) that reversibly catalyzes the phosphorolysis of purine nucleoside to their respective purine bases and the corresponding pentose-1-phosphate. PNP activity can be inhibited by 8-aminoguanosine⁽⁵⁸⁾. Four G-protein coupled receptors, the calcitonin gene-related peptide receptor (CGRP-1), a human homolog of rat RDC1, the endothelin B receptor, and the chemokine receptor (and HIV-coreceptor) CXCR4, were also specifically induced during the lytic cycle. Both RDC1 and CGRP-1 can be inhibited with a truncated version of its ligand, the calcitonin gene-related peptide^{(59, 60)}. Endothelin binding to the endothelin B receptor can be blocked by the antagonist peptide BQ 788⁽⁶¹⁾. Likewise, stromal cell derived factor-1 binding to CXCR4, as well as HIV fusion, can be inhibited by peptide T22. PDGF-mediated induction of MCP-1 can be inhibited with trans-retinoic acid⁽⁵³⁾. Several genes involved in signal transduction were induced during lytic infection. Hsp27 is part of the stress pathway activated by p38MAPK, and the MAPK-mediated phosphorylation of Hsp27 can be inhibited by SB203580. Gravin belongs to a family of anchoring proteins responsible for subcellular localization of protein kinase A⁽⁶³⁾. The interaction between Gravin and PKA can be inhibited with the AKAP-fragment peptide Ht31 both in vitro and in cells^{(64, 65)}. Type I sigma receptor activation augments signaling from the N-methyl-D-aspartate (NMDA) receptor⁽⁶⁶⁾, and its activity can be modulated by the calcitonin gene-related peptide (CGRP) and neuropeptide Y (NPY) receptors⁽⁶⁷⁾. The type 1 sigma receptor is antagonized by the anti-psychotic drug haloperidol.

Cells were treated with inhibitory compounds or peptides for one hour and lytic replication was induced by PMA treatment for 48 hours. Ganciclovir was used as control compound for inhibition of KSHV-replication and late gene expression⁽¹⁰⁾. After 48 hours, cells were harvested and expression of the latent gene K12 as well as the late gene ORF 65 was analyzed by RT-PCR. For control, expression of cellular genes α-actin or HPRT was monitored. As expected Ganciclovir did not interfere with latent gene expression but inhibited ORF 65 transcription (FIG. 7). In contrast, most other inhibitors did not interfere with either KSHV latent or late gene expression. However, both Ht31 and Haloperidol inhibited late gene expression, but not latent gene expression. Since both inhibitors potentially interfere with PKC signaling, we examined whether Ht31 or Haloperidol would inhibit the PMA-mediated induction of immediate early genes. However, neither compound prevented induction of the immediate early gene K3. In addition, the peptide Ht-31 inhibits the association between A kinase anchoring proteins (AKAPs) and PKA (21, 64). 25 μM Ht-31 inhibited ORF65 expression without affecting the level of K12 RNA (FIG. 7B, lane 6). Therefore we conclude that the signal transduction pathways regulated by the type I sigma receptor and AKAPs are required for lytic progression of KSHV.

The PKA-AKAP interaction and the type I Sigma receptor are required for ORF 65 expression. The AKAP Gravin (AKAP250) was initially identified as an autoantigen in patients suffering from myasthenia gravis⁽²⁵⁾. Gravin may be involved in signaling from beta-2 adrenergic receptors⁽⁶⁴⁾, and may mediate cross-talk between the PKA and PKC pathways⁽⁴⁷⁾. To corroborate that Gravin was up-regulated in infected cells, an RT-PCR was performed (FIG. 7A). The PCR analysis confirmed that Gravin was overexpressed in infected cells, the highest level of expression being observed at 6 and 48 hours post-induction (FIG. 7A).

There is no specific inhibitor of Gravin identified, however the Ht-31 peptide derived from the PKA R-II binding domain of the human thyroid AKAP inhibits the AKAP/PKA interactions in vivo and in vitro ^{(21, 64)}. A stearated version of this peptide is membrane permeable and can block the AKAP/PKA interaction in tissue culture. A requirement for PKA signaling can be inferred from the up-regulation of Gravin, and to further investigate the requirement for the AKAP/PKA interaction for KSHV late gene expression, infected and uninfected cells were treated with stearated Ht-31 at two concentrations (FIG. 7B). At both the 25 μM and 5 μM concentrations, Ht-31 efficiently repressed ORF 65 expression to levels below those seen in un-induced cells (FIG. 4B, compare lanes 2-3 with 1). A stearated control peptide that is unable to interact with PKA, Ht-31P, had no effect on late gene expression (FIG. 7B, lanes 4-5). These results were reproducibly obtained using different strains of infected cells. These data demonstrate that PKA signaling is required for expression of the ORF 65 gene. It is not possible to conclude whether the inhibitory effect of Ht-3 1 occurs through Gravin or any other AKAP, since Ht-31 inhibits a wide range of AKAP/PKA interactions.

The type I Sigma receptor is expressed in a wide variety of cell types⁽³¹⁾, and may be involved in regulation of intracellular calcium concentrations. To corroborate that the type I Sigma receptor was up-regulated in infected cells, an RT-PCR assay was performed (FIG. 7C). The RT-PCR analysis confirmed that the Sigma receptor is expressed at low levels in latently infected cells, and significantly induced during lytic replication (FIG. 7C). However, the RT-PCR analysis suggests that the type I Sigma receptor is significantly induced already 6 hours post-induction (FIG. 4C, lane 2), in contrast to the data obtained from the chip. This may reflect a certain variation between experimental conditions, or a variation between different strains of infected cells. However, the data confirms that the Sigma receptor is significantly up-regulated during the lytic phase.

The type I Sigma receptor is antagonized by the antipsychotic drug Haloperidol⁽⁶⁶⁾. To investigate the requirement of Sigma receptor function for late gene expression, cells were treated with 5 μM Haloperidol for 1 hour prior to PMA addition. RT-PCR analysis demonstrated that whereas Haloperidol did not affect expression of the latent K12 gene, ORF 65 expression was significantly reduced (FIG. 7D, lane 5). The inhibition of late gene expression was more complete with Haloperidol than with Ganciclovir (FIG. 7D, lanes 5-6). In contrast, the purine nucleotide phosphorylase inhibitor 8-aminoguanosine (8-AMG) had no effect on viral gene expression (FIG. 7D, lane 4). These data were reproduced using different strains of infected cells thus confirming that the type I Sigma receptor is required for ORF 65 expression. Our results demonstrate that DNA microarray analysis can be used to identify cellular factors required for late gene expression, and thus represents a new tool for antiviral research.

### Example 2

Antisense Therapy Utilizing a c-kit Antisense Oligomer Inhibits c-kit Protein Expression and Prevents Development of the Transformed Phenotype in KSHV-Infected DMVEC.

To investigate the ability of antisense therapy to ameliorate the phenotypic changes associated with KSHV-induced c-kit expression in DMVEC, we designed a phosphorodiamidate antisense (PMO-AS) oligomer targeting the start codon of c-kit to block protein translation. PMO-AS oligomers are thought to inhibit translation by steric hindrance and have the advantage of not activating RNAse H. To facilitate endocytic uptake of the oligomer and delivery to the cytosol, ethoxylated polyethylenimine (EPEI) that forms an anionic complex with the PMO-AS was used as a delivery reagent. A FITC moiety was incorporated into the oligomer to allow monitoring of loading efficiency by fluorescence microscopy.

An initial titration experiment on KSHV-infected DMVEC was performed with a range of different oligomer/EPEI volumes. Loading 1.25 nMol oligomer with 2.5 μl EPEI reagent per 35 mm dish allowed efficient antisense uptake without non-specific EPEI-induced toxicity. To verify that the c-kit oligomer was effective in down-regulating c-Kit, KSHV-infected DMVEC were loaded with the anti-c-kit oligomer-EPEI complex and incubated for 3 hours at 37° C. in serum-free medium to allow for oligomer uptake. As controls, DMVEC cultures were treated with EPEI reagent and sterile water or sterile water alone, or were loaded with an oligomer/EPEI complex containing an irrelevant PITC-tagged oligomer. Upon removal of the oligomer-EPEI solution, cell monolayers were rinsed in serum-free medium, fed with complete medium and examined daily to evaluate oligomer uptake and stability. A strong cytoplasmic fluorescence was observed by day 1 post loading and was well maintained for up to 10 days. On day 4 post loading, cells were fixed and stained with a monoclonal antibody against c-kit and a goat-anti-mouse-Alexa conjugate to evaluate c-kit expression levels. While c-kit expression was strong in cells treated with control oligomer or delivery reagents only, cultures treated with the c-kit oligomer demonstrated significantly decreased c-kit levels. Importantly, occasional individual cells that had not taken up the c-kit oligomer (FITC-negative) still exhibited strong c-kit expression and could be observed adjacent to oligomer-loaded cells (FITC-positive) that consistently displayed negligible c-kit expression. A representative example of this observation is shown in FIG. 8A and strongly suggests that antisense activity is directly responsible for the observed reduction in c-kit expression.

We next ascertained whether antisense therapy was effective in preventing the KSHV-induced transformation in which c-kit overexpression was shown to play a crucial role. For these experiments, KSHV-infected DMVEC were grown to confluence in 35 mm dishes, loaded with antisense as described above, and maintained in a post-confluent state to allow focus formation. This approach was favored over loading of cells that had already formed foci since trial experiments revealed that cells with pre-formed foci were abnormally sensitive to EPEI toxicity. As expected from our previous experiments, post-confluent KSHV-infected DMVEC exhibited loss of contact inhibition and formed disorganized multi-layered foci that were extremely evident by day 6 post-loading (FIG. 8B). In contrast however, cells loaded with 1.25 nMol of the anti-c-kit oligomer and 2.5 μl EPEI did not develop foci but maintained a quiescent contact-inhibited monolayer (FIG. 8C). Cells cultured with 2.5 μl EPEI alone showed similar focus formation to untreated cells (FIG. 8D). Mock-infected KSHV monolayers became quiescent at confluence and did not form foci regardless of antisense treatment, but no treatment-related toxicity was observed. Our previous experiments have demonstrated a direct role for c-kit overexpression in DMVEC transformation. The inability of cultures treated with a c-kit antisense oligomer to develop a transformed phenotype strongly suggests an effective role for antisense in inhibiting c-kit function.

TABLE 1

| Direct Target | Indirect Target | Inhibitor |
|---|---|---|
| NF-kB | Jagged1 | PDTC |
| NF-kB | Heparin-binding EGF-like | PDTC |
| NF-kB | Cyclooxygenase-2 | PDTC |
| NF-kB | A20 | PDTC |
| NF-kB | MMP-10 | PDTC |
| NF-kB | ADAMTS-1 | PDTC |
| NF-kB | IL-8 | PDTC |
| NF-kB | ICAM-1 | PDTC |
| NF-kB | Angiopoietin-2 | PDTC |
| NF-kB | Alpha-1 Integrin | PDTC |
| RAR | PDGF-B | Trans-retinoic acid |
| p38 MAPK | Hsp27 | SB203580 |
| CGRP-1 | | CGRP[8-37] |
| Gravin/AKAPs | | Ht-31 |
| Type I Sigma receptor | | Haloperidol |
| Purine nucleoside | | 8-aminoguanosine |
| phosphorylase | | |
| RDC1 | | CGRP[8-37] |
| CXCR-4 | | t22 |
| Endothelin B receptor | | BQ 788 |

TABLE 2
CELLULAR TRANSCRIPTS CHANGING MORE THAN 1.8 FOLD IN
TWO OUT OF THREE INJECTED CULTURES AT ONE OF EACH TIME POINTS

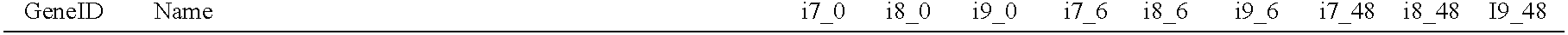

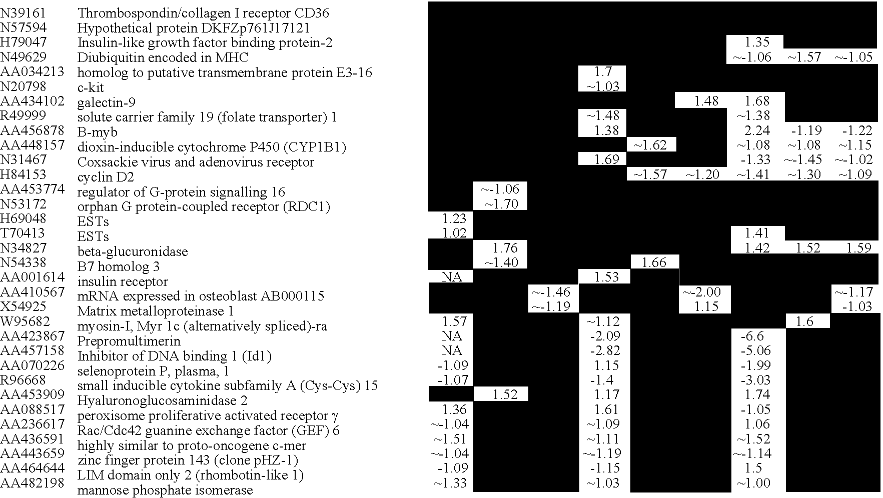

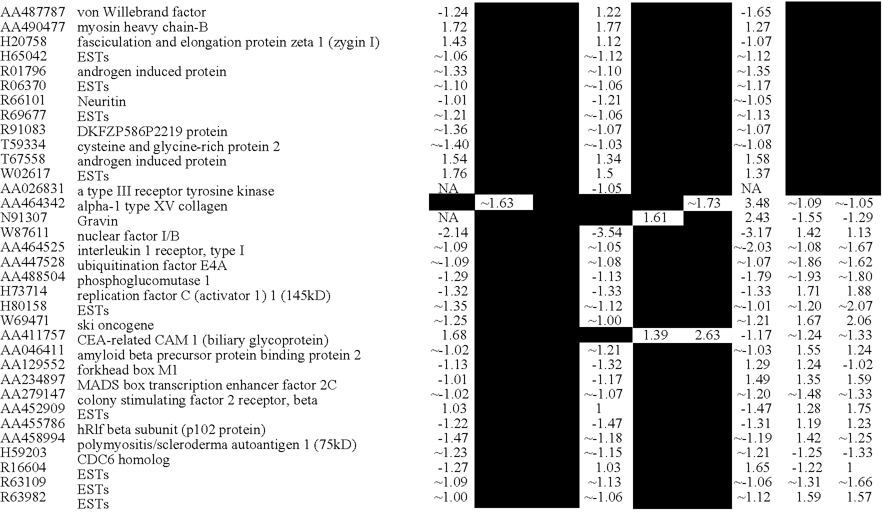

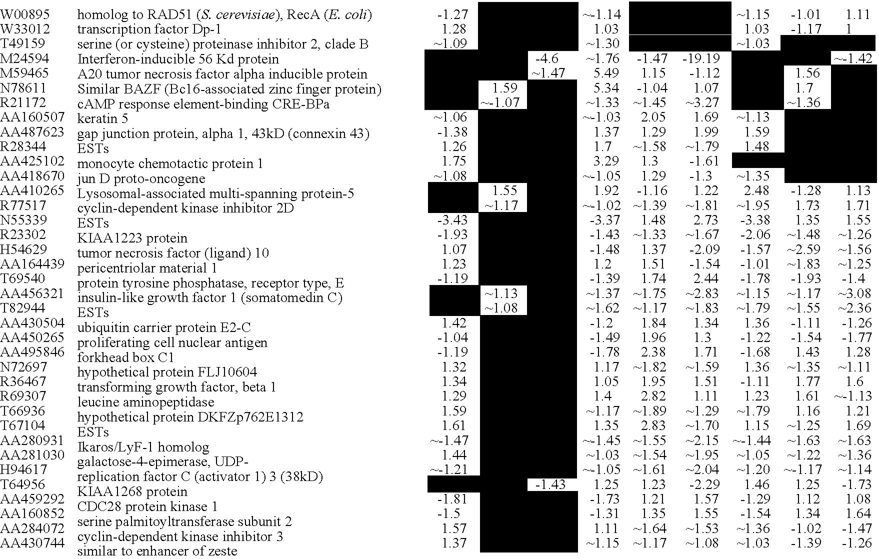

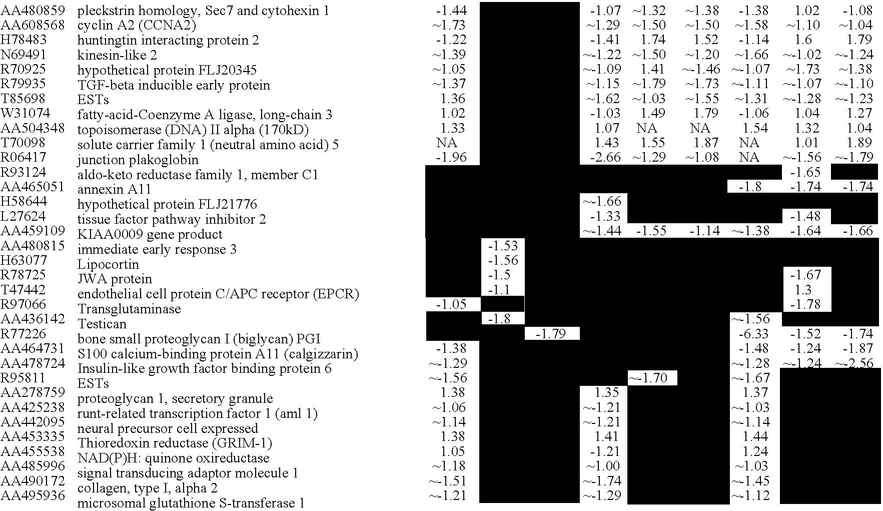

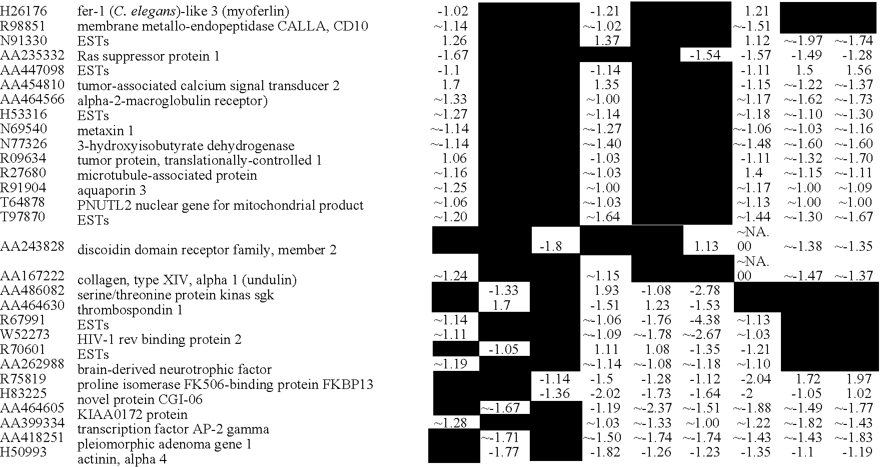

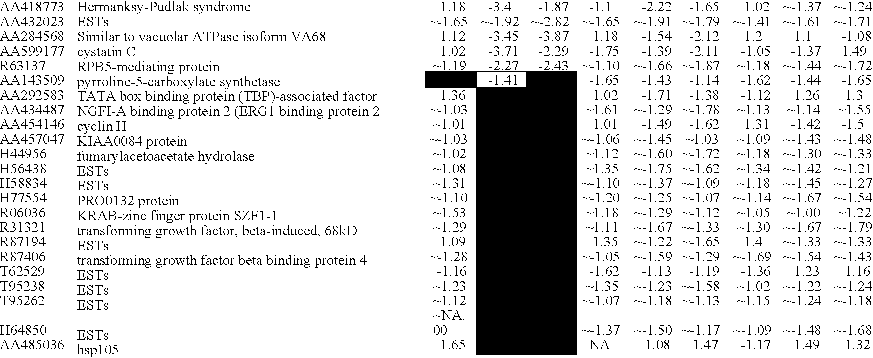

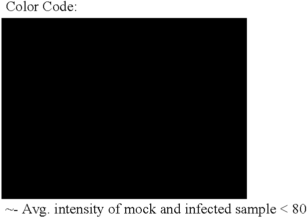

Legend to Table: Shown are the ratios between KSHV-infected endothelial cells and uninfected endothelial cells. Only transcripts that changed more than 1.8 fold (up or down) in at least 2 out of the three infected cell cultures tested at each time point. A ˜ indicates that both the infected and uninfected cell sample had an average intensity below 80 thus indicating a weak signal in both samples.

### REFERENCES

1. Aoki, Y., E. S. Jaffe, Y. Chang, K. Jones, J. Teruya-Feldstein, P. S. Moore, and G. Tosato, “Angiogenesis and Hematopoiesis Induced by Kaposi's Sarcoma Associated Herpesvirus-Encoded Interleukin-6” Blood (1999) 93:4034-43.

2. Aoki, Y., and G. Tosato, “Role of Vascular Endothelial Growth Factor/Vascular Permeability Factor in the Pathogenesis of Kaposi's Sarcoma-Associated Herpesvirus-Infected Primary Effusion Lymphomas” Blood (1999) 94:4247-54.

3. Arvanitakis, L., E. Geras-Raaka, A. Varmna, M. C. Gershengom, and E. Cesarman, “Human Herpesvirus KSHV Encodes a Constitutively Active G-Protein-coupled Receptor Linked to Cell Proliferation” Nature (1997) 385:347-50.

4. Asou, H., J. W. Said, R. Yang, R. Munker, D. J. Park, N. Kamada, and H. P. Koeffler, “Mechanisms of Growth Control of Kaposi's Sarcoma-Associated Herpes Virus-Associated Primary Effusion Lymphoma Cells” Blood (1998) 91:2475-81.

5. Bais, C., B. Santomasso, O. Coso, L. Arvanitakis, E. G. Raaka, J. S. Gutkind, A. S. Asch, E. Cesarman, M. C. Gershengorn, E. A. Mesri, and M. C. Gerhengorn, “G-Protein-Coupled Receptor of Kaposi's Sarcoma-Associated Herpesvirus is a Viral Oncogene and Angiogenesis” [published erratum in Nature 392(6672): 210] Nature (1998) 391:86-9.

6. Bayes-Genis, A., C. A. Conover, and R. S. Schwartz, “The Insulin-like Growth Factor Axis: A Review of Atherosclerosis and Restenosis” Circ. Res. (2000) 86:125-130.

7. Bird, G., G. Burgess, and J. J. Putney, “Sulfhydryl Reagents and cAMP-dependent Kinase Increase the Sensitivity of the Inositol 1,4,5-Triphosphate Receptor in Hepatocytes” J Biol Chem. (1993) 268:17917-17923.

8. Bodo, M., T. Baroni, F. Carinci, E. Becchetti, C. Bellucci, C. Conte, F. Pezzetti, R. Evangelisti, M. Tognon, and P. Carinci, “A Regulatory Role of Fibroblast Growth Factor in the Expression of Decorin, Biglycan, Betaglycan, and Syndecan in Osteoblasts from Patients with Crouzon's Syndrome” Eur. J. Cell Biol. (1999) 78:323-330.

9. Boshoff, C., S. J. Gao, L. E. Healy, S. Matthews, A. J. Thomas, L. Coignet, R. A. Warnke, J. A. Strauchen, E. Matutes, O. W. Karnel, P. S. Moore, R. A. Weiss, and Y. Chang, “Establishing a KSHV+Cell Line (BCP-1) from Peripheral Blood and Characterizing its Growth in nod/SCID mice” Blood (1998) 91:1671-1679.

10. Cannon, J., F. Hamzeh, S. Moore, J. Nicholas, and R. Ambinder, “Human Herpesvirus 8-Encoded Thymidine Kinase and Phosphotransferase Homologues Confer Sensitivity to Ganciclovir” J Virol. (1999) 73:4786-4793.

11. Cesarman, E., Y. Chang, P. S. Moore, J. W. Said, and D. M. Knowles, “Kaposi's Sarcoma-Associated Herpesvirus-like DNA sequences in AIDS-Related Body Cavity-Based Lymphomas” N Engl. J Med. (1995)332:1186-91.

12. Cesarman, E., R. G. Nador, F. Bai, R. A. Bohenzky, J. J. Russo, P. S. Moore, Y. Chang, and D. M. Knowles, “Kaposi's Sarcoma-Associated Herpesvirus Contains G Protein-Coupled Receptor and Cyclin D Homologs which are Expressed in Kaposi's Sarcoma and Malignant Lymphoma” J Virol. (1996) 70:8218-23.

13. Chang, J., R. Renne, D. Dittmer, and D. Ganem, “Inflammatory Cytokines and the Reactivation of Kaposi's Sarcoma-Associated Herpesvirus Lytic Replication” Virology. (2000) 266:17-25.

14. Chang, Y., E. Cesarman, M. S. Pessin, F. Lee, J. Culpepper, D. M. Knowles, and P. S. Moore, “Identification of Herpesvirus-like DNA sequences in AIDS-Associated Kaposi's Sarcoma” Science. (1994) 266:1865-9.

15. Chang, Y. E., and L. A. Laimins, “Microarray Analysis Identifies Interferon-Inducible Genes and Stat-1 as Major Transcriptional Targets of Human Papillomavirus Type 31” J. Virol. (2000) 74:4174-4182.

16. Cheng, E. H., J. Nicholas, D. S. Bellows, G. S. Hayward, H. G. Guo, M. S. Reitz, and J. M. Hardwick, “A Bcl-2 Homolog Encoded by Kaposi Sarcoma-Associated Virus, Human Herpesvirus 8, Inhibits Apoptosis but Does Not Heterodimerize with Bax or Bak” Proc. Natl. Acad. Sci. U S A. (1997) 94:690-4.

17. Chin, Y. E., M. Kitagawa, W. C. S. Su, Z. -H. You, Y. Iwamoto, and X. -Y. Fu, “Cell Growth Arrest and Induction of Cyclin-Dependent Kinase Inhibitor p21WAF1/CIP1 mediated by STAT1” Science (Washington, D.C.) (1996) 272:719-22.

18. Choi, K., M. Kennedy, A. Kazarov, J. C. Papadimitriou, and G. Keller, “A Common Precursor for Hematopoietic and Endothelial Cells” Development (1998) 125:725-32.

19. Djerbi, M., V. Screpanti, A. I. Catrina, B. Bogen, P. Biberfeld, and A. Grandien, “The Inhibitor of Death Receptor Signaling, FLICE-Inhibitory Protein Defines a New Class of Tumor Progression Factors” J. Exp. Med. (1999) 190:1025-32.

20. Dupin, N., C. Fisher, P. Kellam, S. Ariad, M. Tulliez, N. Franck, E. Van Marck, D. Salmon, I. Gorin, J. -P. Escande, R. A. Weiss, K. Alitalo, and C. Boshoff, “Distribution of Human Herpesvirus-8 Latently Infected Cells in Kaposi's Sarcoma, Multicentric Castleman's Disease, and Primary Effusion Lymphoma” Proc. Natl. Acad. Sci. U. S. A. (1999) 96:4546-4551.

21. Edwards, A. S., and J. D. Scott, “A-Kinase Anchoring Proteins: Protein Kinase A and Beyond” Curr. Opin. Cell Biol. (2000) 12:217-221.

22. Ellis, M., Y. P. Chew, L. Fallis, S. Freddersdorf, C. Boshoff, R. A. Weiss, X. Lu, and S. Mittnacht, “Degradation of p27Kip cdk Inhibitor Triggered by Kaposi's Sarcoma Virus Cyclin-cdk6 Complex” EMBO J. (1999) 18:644-653.

23. Flore, O., S. Rafii, S. Ely, J. J. O'Leary, E. M. Hyjek, and E. Cesarman, “Transformation of Primary Human Endothelial Cells by Kaposi's Sarcoma-Associated Herpesvirus” Nature. (1998) 394:588-92.

24. Gao, S. J., C. Boshoff, S. Jayachandra, R. A. Weiss, Y. Chang, and P. S. Moore, “KSHV ORF K9 (vIRF) is an Oncogene which Inhibits the Interferon Signaling Pathway” Oncogene. (1997) 15:1979-85.

25. Gordon, T., B. Grove, J. C. Loftus, T. O'Toole, R. McMillan, J. Lindstrom, and M. H. Ginsberg, “Molecular Cloning and Preliminary Characterization of a Novel Cytoplasmic Antigen Recognized by Myasthenia Gravis Sera” J. Clin. Invest. (1992) 90:992-9.

26. Gray, P., J. Scott, and W. Catterall, “Regulation of Ion Channels by cAMP-dependent Protein Kinase and A-Kinase Anchoring Proteins” Curr. Op. Neurobiol. (1998) 8:330-334.

27. Grimberg, A., and P. Cohen, “Role of Insulin-like Growth Factors and Their Binding Proteins in Growth Control and Carcinogenesis” J. Cell. Physiol. (2000) 183:1-9.

28. Hayashi, T., A. Kagaya, M. Takebayashi, M. Shimizu, Y. Uchitomi, N. Motohashi, and S. Yamawaki, “Modulation by Sigma Ligands of Intracellular Free Ca++ Mobilization by N-methyl-D-Aspartate in Primary Culture of Rat Frontal Cortical Neurons” J Pharmacol. Exp Ther. (1995) 275:207-214.

29. Hayashi, T., T. Maurice, and T. Su, “Ca(2+) Signaling Via Sigma(1)-Receptors: Novel Regulatory Mechanism Affecting Intracellular Ca(2+) Concentration” J Pharmacol. Exp Ther. (2000) 293:788-798.

30. Holash, J., P. C. Maisonpierre, D. Compton, P. Boland, C. R. Alexander, D. Zagzag, G. D. Yancopoulos, and S. J. Wiegand, “Vessel Cooption, Regression, and Growth in Tumors Mediated by Angiopoietins and VEGF” Science (1999) 284:1994-1998.

31. Jbilo, O., H. Vidal, R. Paul, N. De Nys, M. Bensaid, S. Silve, P. Carayon, D. Davi, S. Galiegue, B. Bourrie, J.C. Guillemot, P. Ferrara, G. Loison, J. P. Maffrand, G. Le Fur, and P. Casellas, “Purification and Characterization of the Human SR 31747A-Binding Protein. A Nuclear Membrane Protein Related to Yeast Sterol Isomerase” J. Biol. Chem. (1997) 272:27107-27115.

32. Klette, K., Y. Lin, L. Clapp, M. DeCoster, J. Moreton, and F. Tortella, “Neuroprotective Sigma Ligands Attenuate NMDA and Trans-ACPD-Induced Calcium Signaling in Rat Primary Neurons” Brain Res. (1997) 756:231-240.

33. Leonard, B. E, “The Potential Contribution of Sigma Receptors to Antidepressant Actions” Antidepressants: (1997) 159-172.

34. Li, M., H. Lee, J. Guo, F. Neipel, B. Fleckenstein, K. Ozato, and J. U. Jung, “Kaposi's Sarcoma-Associated Herpesvirus Viral Interferon Regulatory Factor” J. Virol. (1998) 72:5433-5440.

35. Li, M., H. Lee, D. W. Yoon, J. C. Albrecht, B. Fleckenstein, F. Neipel, and J. U.

Jung, “Kaposi's Sarcoma-Associated Herpesvirus Encodes a Functional Cyclin” J Virol. (1997) 71:1984-91.

36. Lowell, B. B, “PPAR.gamma.: An Essential Regulator of Adipogenesis and Modulator of Fat Cell Function” Cell (1999) 99:239-242.

37. Mann, D. J., E. S. Child, C. Swanton, H. Laman, and N. Jones, “Modulation of p27Kip1 Levels by the Cyclin Encoded by Kaposi's Sarcoma-Associated Herpesvirus” EMBO J. (1999) 18:654-663.

38. Marumo, T., V. B. Schini-Kerth, B. Fisslthaler, and R. Busse, “Platelet-Derived Growth Factor-Stimulated Superoxide Anion Production Modulates Activation of Transcription Factor NF-.kappa.B and Expression of Monocyte Chemoattractant Protein 1 in Human Aortic Smooth Muscle Cells” Circulation (1997) 96:2361-2367.

39. Masood, R., J. Cai, T. Zheng, D. L. Smith, Y. Naidu, and P. S. Gill, “Vascular Endothelial Growth Factor/Vascular Permeability Factor is an Autocrine Growth Factor for AIDS-Kaposi Sarcoma” Proc. Natl. Acad. Sci. U S A. (1997) 94:979-84.

40. Meade-Tollin, L. C., D. Way, and M. H. Witte, “Expression of Multiple Matrix Metalloproteinases and Urokinase Type Plasminogen Activator in Cultured Kaposi Sarcoma Cells” Acta Histochem. (1999) 101:305-316.

41. Miles, S. A., A. R. Rezai, J. F. Salazar-Gonzalez, M. Vander Meyden, R. H. Stevens, D. M. Logan, R. T. Mitsuyasu, T. Taga, T. Hirano, T. Kishimoto, and et al, “AIDS Kaposi Sarcoma-Derived Cells Produce and Respond to Interleukin 6” Proc. Natl. Acad. Sci. U S A. (1990) 87:4068-72.

42. Morin-Surun, M. P., T. Collin, M. Denavit-Saubie, E. E. Baulieu, and F. P. Monnet, “Intracellular Sigma.1 Receptor Modulates Phospholipase C and Protein Kinase C Activities in the Brainstem” Proc. Natl. Acad. Sci. U. S. A. (1999) 96:8196-8199.

43. Moses, A. V., K. N. Fish, R. Ruhl, P. P. Smith, J. G. Strussenberg, L. Zhu, B. Chandran, and J. A. Nelson, “Long-Term Infection and Transformation of Dermal Microvascular Endothelial Cells by Human Herpesvirus 8” J. Virol. (1999) 73:6892-6902.

44. Moses, H. L., and R. Serra, “Regulation of Differentiation by TGF-.Beta” Curr. Opin. Genet. Dev. (1996) 6:581-586.

45. Nair, B. C., A. L. DeVico, S. Nakamura, T. D. Copeland, Y. Chen, A. Patel, T. O'Neil, S. Oroszlan, R. C. Gallo, and M. G. Sarngadharan, “Identification of a Major Growth Factor for AIDS-Kaposi's Sarcoma Cells as Oncostatin” M. Science (1992) 255:1430-2.

46. Nakamura, S., K. Murakami-Mori, N. Rao, H. A. Weich, and B. Rajeev, “Vascular Endothelial Growth Factor is a Potent Angiogenic Factor in AIDS-Associated Kaposi's Sarcoma-Derived Spindle Cells” J Immunol. (1997) 158:4992-5001.

47. Nauert, J. B., T. M. Klauck, L. K. Langeberg, and J. D. Scott, “Gravin, An Autoantigen Recognized by Serum from Myasthenia Gravis Patients, is a Kinase Scaffold Protein” Curr. Biol. (1997) 7:52-62.

48. Neipel, F., J. C. Albrecht, A. Ensser, Y. Q. Huang, J. J. Li, A. E. Friedman-Kien, and B. Fleckenstein, “Human Herpesvirus 8 Encodes a Homolog of Interleukin-6” J Virol. (1997) 71:839-42.

49. Nemerow, G. R., and N. R. Cooper, “Infection of B Lymphocytes by a Human Herpesvirus, Epstein-Barr Virus, is Blocked by Calmodulin Antagonists” Proc. Natl. Acad. Sci. U. S. A. (1984) 81:4955-9.

50. Nicholas, J., V. Ruvolo, J. Zong, D. Ciufo, H. G. Guo, M. S. Reitz, and G. S.

Hayward, “A Single 13-Kilobase Divergent Locus in the Kaposi Sarcoma-Associated Herpesvirus (Human Herpesvirus 8) Genome Contains Nine Open Reading Frames that are Homologous to or Related to Cellular Proteins” J Virol. (1997) 71:1963-74.

51. Ohtomo, T., Y. Sugamata, Y. Ozaki, K. Ono, Y. Yoshimura, S. Kawai, Y. Koishihara, S. Ozaki, M. Kosaka, T. Hirano, and M. Tsuchiya, “Molecular Cloning and Characterization of a Surface Antigen Preferentially Overexpressed on Multiple Myeloma Cells” Biochem. Biophys. Res. Commun. (1999) 258:583-591.

52. Okabe, S., T. Fukuda, K. Ishibashi, S. Kojima, S. Okada, M. Hatano, M. Ebara, H. Saisho, and T. Tokuhisa, “BAZF, A Novel Bcl6 Homolog, Functions as a Transcriptional Repressor” Mol. Cell. Biol. (1998) 18:4235-4244.

53. Ping, D., G. Boekhoudt, and J. M. Boss, “Trans-Retinoic Acid Blocks Platelet-Derived Growth Factor-bb-Induced Expression of the Murine Monocyte Chemoattractant-1 Gene by Blocking the Assembly of a Promoter Proximal Sp1 Binding Site” J. Biol. Chem. (1999) 274:31909-31916.

54. Pittenger, M. F., A. M. Mackay, S. C. Beck, R. K. Jaiswal, R. Douglas, J. D. Mosca, M. A. Moorman, D. W. Simonetti, S. Craig, and D. R. Marshak, “Multilineage Potential of Adult Human Mesenchymal Stem Cells” Science. (1999) 284:143-147.

55. Roth, W. K., S. Werner, C. G. Schirren, and P. H. Hofschneider, “Depletion of PDGF from Serum Inhibits Growth of AIDS-Related and Sporadic Kaposi's Sarcoma Cells in Culture” Oncogene. (1989) 4:483-7.

56. Russo, J. J., R. A. Bohenzky, M. C. Chien, J. Chen, M. Yan, D. Maddalena, J. P. Parry, D. Peruzzi, I. S. Edelman, Y. Chang, and P. S. Moore, “Nucleotide Sequence of the Kaposi Sarcoma-Associated Herpesvirus (HHV8)” Proc Natl Acad Sci U S A. (1996) 93:14862-7.

57. Salunga, R. C., H. Guo, L. Luo, A. Bittner, K. C. Joy, J. R. Chambers, J. S. Wan, J. R. Jackson, and M. G. Erlander, “Gene Expression Analysis Via cDNA Microarrays of Laser Capture Microdissected Cells from Fixed Tissue” (1999) In M. Schena (ed.), DNA Microarrays. A Practical Approach. Oxford Press, Oxford, pp. 121-136.

58. Sarid, R., O. Flore, R. A. Bohenzky, Y. Chang, and P. S. Moore, “Transcription Mapping of the Kaposi's Sarcoma-Associated Herpesvirus (Human Herpesvirus 8) Genome in a Body Cavity-Based Lymphoma Cell Line (BC-1)” J. Virol. (1998) 72:1005-1012.

59. Sarid, R., T. Sato, R. A. Bohenzky, J. J. Russo, and Y. Chang, “Kaposi's Sarcoma-Associated Herpesvirus Encodes a Functional bcl-2 homologue” Nat Med. (1997) 3:293-8.

60. Schena, M., D. Shalon, R. W. Davis, and P. O. Brown, “Quantitative Monitoring of Gene Expression Patterns with a Complementary DNA Microarray” Science. (1995) 270:467-70.

61. Schreck, R., B. Meier, D. Mannel, W. Droge, and P. Baeuerle, “Dithiocarbamates as Potent Inhibitors of Nuclear Factor Kappa B Activation in Intact Cells” J Exp Med. (1992) 175:1181-1194.

62. Sciacca, F. L., M. Stuerzl, F. Bussolino, M. Sironi, H. Brandstetter, C. Zietz, D.

Zhou, C. Matteucci, G. Peri, and et al, “Expression of Adhesion Molecules, Platelet-Activating Factor, and Chemokines by Kaposi's Sarcoma Cells” J. Immunol. (1994) 153:4816-25.

63. Shaywitz, A., and M. Greenberg, “CREB: A Stimulus-Induced Transcription Factor Activated by a Diverse Array of Extracellular Signals” Ann Rev Biochem. (1999) 68:821-861.

64. Shih, M., F. Lin, J. D. Scott, H. -Y. Wang, and C. C. Malbon, “Dynamic Complexes of Beta2 Adrenergic Receptors with Protein Kinases and Phosphatases and the Role of Gravin” J. Biol. Chem. (1999) 274:1588-1595.

65. Soulier, J., L. Grollet, E. Oksenhendler, P. Cacoub, D. Cazals-Hatem, P. Babinet, M. F. d'Agay, J. P. Clauvel, M. Raphael, L. Degos, and et al, “Kaposi's Sarcoma-Associated Herpesvirus-like DNA Sequences in Multicentric Castleman's Disease” Blood (1995) 86:1276-80.

66. Su, T., S. Schell, F. Ford-Rice, and E. London, “Correlation of Inhibitory Potencies of Putative Antagonists for Sigma Receptors in Brain and Spleen” Eur. J. Pharmacol. (1988) 148:467-470.

67. Swanton, C., D. J. Mann, B. Fleckenstein, F. Neipel, G. Peters, and N. Jones, “Herpes Viral Cyclin/cdk6 Complexes Evade Inhibition by cdk Inhibitor Proteins” Nature (1997) 390:184-187.

68. Taraboletti, G., R. Benelli, P. Borsotti, M. Rusnati, M. Presta, R. Giavazzi, L. Ruco, and A. Albini, “Thrombospondin-1 Inhibits Kaposi's Sarcoma (KS) Cell and HIV-1 Tat-Induced Angiogenesis and is Poorly Expressed in KS Lesions” J. Pathol. (1999) 188:76-81.

69. Wellstein, A., R. Lupu, G. Zugmaier, S. L. Flamm, A. L. Cheville, P. Delli Bovi, C. Basilico, M. E. Lippman, and F. G. Kern, “Autocrine Growth Stimulation by Secreted Kaposi Fibroblast Growth Factor but not by Endogenous Basic Fibroblast Growth Factor” Cell Growth Differ (1990) 1:63-71.

70. Westphal, R., S. Tavalin, J. Lin, N. Alto, I. Fraser, L. Langeberg, M. Sheng, and J. Scott, “Regulation of NMDA Receptors by an Associated Phosphatase-Kinase Signaling Complex” Science (1999) 285:93-96.

71. Zhang, Q. Y., C. Hammerberg, J. J. Baldassare, P. A. Henderson, D. Burns, M. Ceska, J. J. Voorhees, and G. J. Fisher, “Retinoic Acid and Phorbol Ester Synergistically Up-Regulate IL-8 Expression and Specifically Modulate Protein Kinase C-.epsilon in Human Skin Fibroblasts” J. Immunol. (1992) 149:1402-8.

72. Zhong, W., H. Wang, B. Herndier, and D. Ganem, “Restricted Expression of Kaposi Sarcoma-Associated Herpesvirus (Human Herpesvirus 8) Genes in Kaposi Sarcoma” Proc. Natl. Acad. Sci. U. S. A. (1996) 93:6641-6646.

73. Zhu, H., J. P. Cong, G. Mamtora, T. Gingeras, and T. Shenk, “Cellular Gene Expression Altered by Human Cytomegalovirus: Global Monitoring with Oligonucleotide Arrays” Proc. Natl. Acad. Sci. U S A. (1998) 95:14470-5.

74. Zimring, J. C., S. Goodbourn, and M. K. Offermann, “Human Herpesvirus 8 Encodes an Interferon Regulatory Factor (IRF) Homolog that Represses IRF-1-Mediated Transcription” J. Virol. (1998) 72:701-707.

75. Raab, G. and M. Klagsbrun “Heparin-binding EGF-like Growth Factor” Biochim. Biophys. Acta. (1997) 1333:F179-F200.

76. Kekuda, R., P. D. Prasad, Y. J. Fei, F. H. Leibach, and V. Ganapathy “Cloning and Functional Expression of the Human Type 1 Sigma Receptor (hSigmaR1)” Biochem. Biophys. Res. Commun. (1996) 229: 553-558.

77. Heinrich, M. C., D. J. Griffith, B. J. Druker, C. L. Wait, K. A. Ott And A. J. Zigler “Inhibition of C-Kit Receptor Tyrosine Kinase Activity by STI 571, a Selective Tyrosine Kinase Inhibitor [In Process Citation]” (2000) Blood 96:925-32.

78. Boshoff, C., T. F. Schulz, M. M. Kennedy, A. K. Graham, C. Fisher, A. Thomas, J. O. McGee, R. A. Weiss, and J. J. O'Leary, “Kaposi's Sarcoma-Associated Herpesvirus Infects Endothelial and Spindle Cells” (1995) Nat Med 1: 1274-8.

79. Li, J. J., Y. Q. Huang, C. J. Cockerell, and A. E. Friedman-Kien, “Localization of Human Herpes-Like Virus Type 8 in Vascular Endothelial Cells and Perivascular Spindle-Shaped Cells of Kaposi's Sarcoma Lesions by In Situ Hybridization” (1996) Am J Pathol. 148:1741-8.

80. Staskus, K. A., W. Zhong, K. Gebhard, B. Herndier, H. Wang, R. Renne, J. Beneke, J. Pudney, D. J. Anderson, D. Ganem, A. T. and Haase, “Kaposi's Sarcoma-Associated Herpesvirus Gene Expression in Endothelial (Spindle) Tumor Cells” (1997) J Virol. 71:715-9.

81. Sturzl, M., C. Blasig, A. Schreier, F. Neipel, C. Hohenadl, E. Cornali, G. Ascherl, S. Esser, N. H. Brockmeyer, M. Ekman, E. E. Kaaya, E. Tschachler, and P. Biberfeld, “Expression of HHV-8 Latency-Associated T0.7 RNA in Spindle Cells and Endothelial Cells of AIDS-Associated, Classical and African Kaposi's Sarcoma” (1997) Int J Cancer 72:68-71.

82. Rainbow, L., G. M. Platt, G. R. Simpson, R. Sarid, S. J. Gao, H. Stoiber, C. S. Herrington, P. S. Moore, and T. F. Schulz, “The 222- to 234-Kilodalton Latent Nuclear Protein (LNA) of Kaposi's Sarcoma-Associated Herpesvirus (Human Herpesvirus 8) is Encoded by Orf 73 and is a Component of the Latency-Associated Nuclear Antigen” (1997) J Virol 71:50915-21.

83. Yarden, Y., W. J. Kuang, T. Yang-Feng, L. Coussens, S. Munemitsu, T J. Dull, E. Chen, J. Schlessinger, U. Francke, and A. Ullrich, “Human Proto-Oncogene c-kit: A New Cell Surface Receptor Tyrosine Kinase for an Unidentified Ligand” (1987) Embo J 6:3341-51.

84. Martin, F. H., S. V. Suggs, K. E. Langley, H. S. Lu, J. Ting, K. H. Okino, C. F. Morris, I. K. McNiece, F. W. Jacobsen, E. A. Mendiaz, and et al., “Primary Structure and Functional Expression of Rat and Human Stem Cell Factor DNAs” (1990) Cell 63:203-11.

85. Hirota, S., K. Isozaki, Y. Moriyama, K. Hashimoto, T. Nishida, S. Ishiguro, K. Kawano, M. Hanada, A. Kurata, M. Takeda, G. Muhammad Tunio, Y. Matsuzawa, Y. Kanakura, Y. Shinomura, and Y. Kitamura, “Gain-of-Function Mutations of c-kit in Human Gastrointestinal Stromal Tumors” (1998) Science 279:577-80.

86. Nishida, T., S. Hirota, M. Taniguchi, K. Hashimoto, K. Isozaki, H. Nakamura, Y. Kanakura, T. Tanaka, A. Takabayashi, H. Matsuda, H., and Y. Kitamura, “Familial Gastrointestinal Stromal Tumours with Germline Mutation of the KIT Gene” [letter] (1998) Nat Genet 19:323-4.

87. Tian, Q., H. F. Frierson, Jr., G. W. Krystal, and C. A. Moskaluk, “Activating c-kit Gene Mutations in Human Germ Cell Tumors” (1999) Am J Pathol 154:1643-7.

88. Lux, M. L., B. P. Rubin, T. L. Biase, C. J. Chen, T. Maclure, G. Demetri, S. Xiao, S. Singer, C. D. Fletcher, and J. A. Fletcher, “KIT Extracellular and Kinase Domain Mutations in Gastrointestinal Stromal Tumors” (2000) Am J Pathol 156:791-5.

89. Turner, A. M., K. M. Zsebo, F. Martin, F. W. Jacobsen, L. G. Bennett, and V. C. Broudy, “Nonhematopoietic Tumor Cell Lines Express Stem Cell Factor and Display c-kit Receptors” (1992) Blood 80:374-81.

90. Matsuda, R., T. Takahashi, S. Nakamura, Y. Sekido, K. Nishida, M. Seto, T. Seito, T. Sugiura, Y. Ariyoshi, and et al., “Expression of the c-kit Protein in Human Solid Tumors and in Corresponding Fetal and Adult Normal Tissues” (1993) Am J Pathol 142:339-46.

91. Inoue, M., S. Kyo, M. Fujita, T. Enomoto, and G. Kondoh, “Coexpression of the c-kit Receptor and the Stem Cell Factor in Gynecological Tumors” (1994) Cancer Res 54:3049-53.

92. Hines, S. J., C. Organ, M. J. Kornstein, and G. W. Krystal, “Coexpression of the c-kit and Stem Cell Factor Genes in Breast Carcinomas” (1995) Cell Growth Differ 6:769-79.

93. Krystal, G. W., S. J. Hines, and C. P. Organ, “Autocrine Growth of Small Cell Lung Cancer Mediated by Coexpression of c-kit and Stem Cell Factor” (1996) Cancer Res 56:370-6.

94. Miettinen, M., M. Sarlomo-Rikala, and J. Lasota, “KIT Expression in Angiosarcomas and Fetal Endothelial Cells: Lack of Mutations of Exon 11 and Exon 17 of c-kit” (2000) Mod Pathol 13:536-41.

95. Druker, B. J., and N. B. Lydon, “Lessons Learned from the Development of an abl Tyrosine Kinase Inhibitor for Chronic Myelogenous Leukemia” (2000) J Clin. Invest 105:3-7.

96. Wang, W. L., M. E. Healy, M. Sattler, S. Verma, J. Lin, G. Maulik, C. D. Stiles, J. D. Griffin, B. E. Johnson, and R. Salgia, “Growth Inhibition and Modulation of Kinase Pathways of Small Cell Lung Cancer Cell Lines by the Novel Tyrosine Kinase Inhibitor STI 571” (2000) Oncogene 19:3521-8.

97. Buchdunger, E., J. Zimmermann, H. Mett, T. Meyer, M. Muller, B. J. Druker, and N. B. Lydon, “Inhibition of the Abl Protein-Tyrosine Kinase In Vitro and In Vivo by a 2-Phenylaminopyrimidine Derivative” (1996) Cancer Res 56:100-4.

98. Druker, B. J., S. Tamura, E. Buchdunger, S. Ohno, G. M. Segal, S. Fanning, J. Zimmermann, and N. B. Lydon, “Effects of a Selective Inhibitor of the abl Tyrosine Kinase on the Growth of bcr-abl Positive Cells” (1996) Nat Med 2:561-6.

99. Streblow, D. N., Soderberg-Naucler, C., Vieira, J., Smith, P., Wakabayashi, E., Ruchti, F., Mattison, K., Altschuler, Y., and Nelson, J. A. (1999). “The Human Cytomegalovirus Chemokine Receptor US28 Mediates Vascular Smooth Muscle Cell Migration” (1999) Cell 99:511-20.

100. Aye, M. T., S. Hashemi, B. Leclair, A. Zeibdawi, E. Trudel, M. Halpenny, V. Fuller, and G. Cheng, “Expression of Stem Cell Factor and C-Kit mRNA in Cultured Endothelial Cells, Monocytes and Cloned Human Bone Marrow Stromal Cells (CFU-RF)” (1992) Exp Hematol 20:523-7.

101. Broudy, V. C., N. L. Kovach, L. G. Bennett, N. Lin, F. W. Jacobsen, and P. G. Kidd, “Human Umbilical Vein Endothelial Cells Display High-Affinity c-kit Receptors and Produce a Soluble Form of the c-kit Receptor” (1994) Blood 83:2145-52.

102. Buzby, J. S., E. M. Knoppel, and M. S. Cairo, “Coordinate Regulation of Steel Factor, its Receptor (Kit), and Cytoadhesion Molecule (ICAM-1 and ELAM-1) mRNA Expression in Human Vascular Endothelial Cells of Differing Origins” (1994) Exp Hematol 22:122-9.

103. Yamaguchi, H., E. Ishii, S. Saito, K. Tashiro, I. Fujita, S. Yoshidomi, M. Ohtubo, K. Akazawa, and S. Miyazaki, “Umbilical Vein Endothelial Cells are an Important Source of C-Kit and Stem Cell Factor Which Regulate the Proliferation of Haemopoietic Progenitor Cells” (1996) Br J Haematol. 94:606-11.

104. Flanagan, J. G., D. C. Chan, and P. Leder, “Transmembrane Form of the Kit Ligand Growth Factor is Determined by Alternative Splicing and is Missing in the Sld Mutant” (1991) Cell 64:1025-35.

105. Heinrich, M. C., D. C. Dooley, A. C. Freed, L. Band, M. E. Hoatlin, W. W. Keeble, S. T. Peters, K. V. Silvey, F. S. Ey, D. Kabat, and et al., “Constitutive Expression of Steel Factor Gene by Human Stromal Cells” (1993) Blood 82:771-83.

106. Buchdunger, E., J. Zimmermann, H. Mett, T. Meyer, M. Muller, U. Regenass, and N. B. Lydon, “Selective Inhibition of the Platelet-Derived Growth Factor Signal Transduction Pathway by a Protein-Tyrosine Kinase Inhibitor of the 2-Phenylaminopyrimidine Class” (1995) Proc. Natl. Acad. Sci. U S A 92:2558-62.

107. Lev, S., Y. Yarden, and D. Givol, “Receptor Functions and Ligand-Dependent Transforming Potential of a Chimeric Kit Proto-Oncogene” (1990) Mol Cell Biol 10:6064-8.

108. Alexander, W. S., S. D. Lyman, and E. F. Wagner, “Expression Of Functional C-Kit Receptors Rescues The Genetic Defect of W Mutant Mast Cells” (1991) Embo. J. 10:3683-91.

109. Caruana, G., A. C. Cambareri, T. J. Gonda, and L. K. Ashman, “Transformation of NIH3T3 Fibroblasts by the c-kit Receptor Tyrosine Kinase: Effect of Receptor Density and Ligand-Requirement” (1998) Oncogene 16:179-90.

110. Chan, S. R., C. Bloomer, and B. Chandran, “Identification and Characterization of Human Herpesvirus-8 Lytic Cycle-Associated ORF 59 Protein and the Encoding cDNA by Monoclonal Antibody” (1998) Virology 240:118-26.

111. Chandran, B., C. Bloomer, S. R. Chan, L. Zhu, E. Goldstein, and R. Horvat, “Human Herpesvirus-8 ORF K8.1 Gene Encodes Immunogenic Glycoproteins Generated by Spliced Transcripts” (1998) Virology 249:140-9.

112. Roehm, N. W., G. H. Rodgers, S. M. Hatfield, and A. L. Glasebrook, “An Improved Colorimetric Assay for Cell Proliferation and Viability Utilizing the Tetrazolium Salt XTT” (1991) J Immunol Methods 142:257-65.

113. Renne, R., D. Blackbourn, D. Whitby, J. Levy, and D. Ganem, “Limited Transmission of Kaposi's Sarcoma-Associated Herpesvirus in Cultured Cells” (1998) J Virol. 72:5182-8.

1-8. (canceled)

9. A method for identifying modulators of Kaposi's sarcoma herpesvirus (KSHV) replication, comprising:
selecting a gene product from a group of genes consisting of the genes listed in Table 2;
combining a test compound with the gene product encoded by the gene;
determining whether the test compound inhibits or activates the gene product;
combining the test compound with KSHV infected cells; and
determining whether the test compound inhibits or activates replication of the KSHV.

10-24. (canceled)

25. The method of claim 9 wherein the gene product is c-kit tyrosine kinase.

26. The method of claim 9 further comprising the step of measuring a change within the cell in response to inhibition or activation of the gene product by the test compound.

27. The method of claim 9 wherein the cells are endothelial cells.

28. The method of claim 27 wherein the cells are dermal microvascular endothelial cells.

29. A method for identifying modulators of Kaposi's sarcoma herpesvirus (KSHV) replication comprising:
contacting a test compound with a functional gene product encoded by a KSHV-modulated endothelial cell gene; and
measuring a modified function of the gene product by the test compound to identify whether the test compound is a modulator of KSHV replication.

30. The method of claim 29 wherein the KSHV-modulated endothelial cell gene is selected from a group of genes listed in Table 2.

31. The method of claim 30 wherein the gene product is c-kit tyrosine kinase.
